(19) United States
(12) Patent Application Publication
(10) Pub. No.: US 2018/0071215 A1
(43) Pub. Date: Mar. 15, 2018

(54) CORE-SHELL PARTICLES FOR CONTROLLED RELEASE

(71) Applicant: Massachusetts Institute of Technology, Cambridge, MA (US)

(72) Inventors: Bernhardt Levy Trout, Lexington, MA (US); Patrick S. Doyle, Sudbury, MA (US); Allan S. Myerson, Boston, MA (US); Paul Douglas Godfrin, Boston, MA (US); Abu Zayed Md Badruddoza, Quincy, MA (US)

(73) Assignee: Massachusetts Institute of Technology, Cambridge, MA (US)

(21) Appl. No.: 15/713,508

(22) Filed: Sep. 22, 2017

Related U.S. Application Data
(63) Continuation-in-part of application No. 14/575,069, filed on Dec. 18, 2014.
(60) Provisional application No. 62/399,290, filed on Sep. 23, 2016.
(60) Provisional application No. 61/917,554, filed on Dec. 18, 2013.

(51) Int. Cl. A61K 9/107; A61K 47/69; A61K 47/36; A61K 9/16; A61K 9/14

(52) U.S. Cl. CPC A61K 9/1075; A61K 47/6907; A61K 47/6933; A61K 9/1617; A61K 9/1652; A61K 9/146; A61K 47/36

(57) ABSTRACT

Compositions, methods, and systems for controlling crystallization of an agent are generally described. In some embodiments, an agent is crystallized in the presence of polymer matrices, such as polymer particles. The polymer matrix may influence at least a portion of the crystallization process and/or the resulting composition. In some such embodiments, the polymer matrix allows one or more aspect of the process and/or composition to be controlled and/or altered. For instance, the polymer matrix may act as a crystallization promoter and/or acceptable carriers of the crystallized agent. In certain embodiments, the polymer matrix described herein, can be used with any agent regardless of its chemical and/or physical properties.

### RELATED APPLICATIONS

The present application is a continuation-in-part of U.S. patent application Ser. No. 14/575,069, filed on Dec. 18, 2014 and entitled “Polymer Matrices for Controlling Crystallization”, which claims the benefit of U.S. Provisional Patent Application Ser. No. 61/917,554, filed Dec. 18, 2013, entitled “Polymer Matrices for Controlling Crystallization,” each of which is incorporated herein by reference in its entirety.

The present application also claims the benefit of U.S. Provisional Patent Application Ser. No. 62/399,290, filed Sep. 23, 2016, entitled “Core-Shell Particles for Controlled Release”, which is incorporated herein by reference in its entirety.

### TECHNICAL FIELD

Compositions, methods, and systems for controlling crystallization of an agent (e.g., pharmaceutically active agent) are generally described.

### BACKGROUND

Crystalline materials are omnipresent in nature, consumer products, and industrial products and practices. Many crystalline materials are formed via a crystallization process. In numerous areas of science and technology, such as the production of pharmaceuticals and other chemicals, the ability to control crystallization is desired. One method for controlling crystallization is to target one or more step in the crystallization process. Most crystallization processes start with heterogeneous nucleation, which occurs at preferential nucleation sites and is a critical step in the crystallization process. However, the process of heterogeneous nucleation, is complex and not well understood.

Accordingly, improved compositions and methods for controlling crystallization are needed.

### SUMMARY

Compositions, methods, and systems for controlling crystallization of an agent are provided. The subject matter of the present invention involves, in some cases, interrelated products, alternative solutions to a particular problem, and/or a plurality of different uses of one or more systems and/or articles.

In one aspect compositions are provided. In some embodiments, the composition comprises a polymer particle comprising crystals of a pharmaceutically active agent, wherein the crystals have an average diameter that is greater than an average mesh size of the polymer particle and wherein the average diameter has a coefficient of variation less than or equal to about 10%.

In certain embodiments, the composition comprises crystals of a pharmaceutically active agent dispersed throughout a cross-linked polymer matrix, wherein the solubility of a polymer matrix precursor in a solvent prior to cross-linking is at least 2 times greater than the solubility of the pharmaceutically active agent in the solvent.

In another aspect, a pharmaceutical composition is provided. In some embodiments, the pharmaceutical composition comprises a particulate polymer carrier and a pharmaceutically active agent primarily encapsulated by the particulate polymer carrier, wherein the active agent has been crystallized in the presence of the particulate polymer carrier.

Certain aspects are related to methods. In some embodiments, the method comprises forming an emulsion comprising a non-aqueous carrier containing a hydrophobic drug dispersed in an aqueous carrier containing a cross-linkable agent, while essentially simultaneously at least partially cross-linking the agent sufficient to form a gel; and removing sufficient aqueous carrier, while essentially simultaneously removing sufficient non-aqueous carrier thereby crystallizing the drug as a dispersion of solid hydrophobic drug in a cross-linked polymeric hydrophilic matrix.

According to certain embodiments, the method comprises forming an emulsion comprising a non-aqueous carrier containing a hydrophobic drug dispersed in an aqueous carrier, wherein the aqueous carrier comprises a first polymer, and wherein the emulsion further comprises poly(vinyl alcohol); at least partially cross-linking the first polymer by exposing it to a cross-linking agent; and removing sufficient aqueous carrier and sufficient non-aqueous carrier, thereby crystallizing the drug as a dispersion of solid hydrophobic drug in a cross-linked matrix comprising the first polymer.

In accordance with some embodiments, the method comprises forming an emulsion comprising a non-aqueous carrier containing a hydrophobic drug dispersed in an aqueous carrier, wherein the aqueous carrier comprises a first polymer, and wherein the emulsion further comprises a second polymer; at least partially cross-linking the first polymer by exposing it to a cross-linking agent; removing sufficient aqueous carrier and the non-aqueous carrier, thereby: crystallizing the drug as a dispersion of solid hydrophobic drug in a cross-linked matrix comprising the first polymer, and forming a composite core-shell particle, wherein the shell comprises the second polymer.

In one aspect, a composite core-shell particle is described. In some embodiments, the composite core-shell particle comprises a core, which comprises a crystalline hydrophobic drug embedded in a cross-linked polymer; and a shell, which comprises poly(vinyl alcohol).

In another aspect crystallization methods are provided. In some embodiments, the method comprises crystallizing a pharmaceutically active agent in a fluid droplet within a polymer particle.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic illustration of a method of crystallizing an agent, according to some embodiments;

FIG. 1B is a schematic illustration of a method of crystallizing an agent, according to some embodiments;

FIG. 2A is a schematic illustration of a method for forming a cross-linked emulsion according to certain embodiments;

FIG. 2B is, according to certain embodiments, a schematic illustration of a method for removing an aqueous carrier and a non-aqueous carrier from a cross-linked emulsion;

FIG. 2C is, in accordance with some embodiments, a schematic illustration of a crystalline solid hydrophobic drug dispersed in cross-linked polymer matrix;

FIG. 3A is a schematic illustration of a method of crystallizing an agent, according to certain embodiments;

FIG. 3B is a schematic illustration of a method of crystallizing an agent, according to certain embodiments;

FIG. 4 is a schematic depiction of an exemplary composite core-shell particle;

FIG. 5 is an illustration of methods of associating an agent with a polymer matrix, according to some embodiments;

FIG. 6A is a graph of rheological measurements, according to one set of embodiments;

FIG. 6B is a graph of the average polymer matrix mesh size versus concentration, according to one set of embodiment;

FIG. 7 is a graph of nucleation induction probability for various polymer matrix formulations, according to certain embodiments;

FIG. 8A is a graph of agent loading for various polymer matrix formulations, according to one set of embodiments;

FIG. 8B is a graph of agent loading for various polymer matrix formulations, according to one set of embodiments;

FIG. 9A is a schematic of an emulsion, according to certain embodiments;

FIG. 9B is an image of an emulsion, according to certain embodiments;

FIG. 9C is an image of polymer matrix, according to certain embodiments;

FIG. 10A is a graph of agent loading, according to one set of embodiments;

FIG. 10B is a graph of agent loading, according to one set of embodiments;

FIG. 11A is a schematic of a crystallization method, according to certain embodiments;

FIG. 11B are images of a polymer matrix at various stages, according to certain embodiments;

FIG. 12A is a graph of X-ray diffraction data, according to one set of embodiments;

FIG. 12B is a graph of differential scanning calorimetry data, according to one set of embodiments;

FIG. 12C is a graph of mean crystal size for various droplet diameters, according to one set of embodiments;

FIG. 12D is a graph of a droplet diameter at various agent concentrations, according to one set of embodiments;

FIG. 13A is a graph of crystal dissolution, according to certain embodiments;

FIG. 13B is a graph of crystal dissolution, according to certain embodiments;

FIG. 14 is a graph of crystal dissolution, according to certain embodiments;

FIG. 15 is, according to certain embodiments, an illustration of an approach for forming core-shell hydrogel particles

FIG. 16A shows an SEM image of composite core-shell hydrogels, according to certain embodiments;

FIG. 16B shows an SEM image of composite core-shell hydrogels, according to certain embodiments;

FIG. 16B shows an SEM image of composite core-shell hydrogels, according to certain embodiments;

FIG. 16C shows an SEM image of composite core-shell hydrogels, according to certain embodiments;

FIG. 16D shows an SEM image of composite core-shell hydrogels, according to certain embodiments;

FIG. 16E shows an SEM image of composite core-shell hydrogels, according to certain embodiments;

FIG. 16F shows an SEM image of composite core-shell hydrogels, according to certain embodiments;

FIG. 17A shows Raman spectra of composite hydrogel formulations, according to certain embodiments;

FIG. 17B shows Raman spectra of composite hydrogel formulations, according to certain embodiments;

FIG. 18 shows XRD patterns of a dried composite hydrogel formulation, according to one set of embodiments;

FIG. 19 shows DSC thermograms of a dried composite hydrogel formulation, in accordance with some embodiments;

FIG. 20 shows the mean crystal size and the mean droplet size plotted as a function of volume fraction, in accordance with certain embodiments;

FIG. 21 shows, according to certain embodiments, a scaling relationship between shell thickness of composite core-shell hydrogel beads and formulation parameters;

FIG. 22 shows, in accordance with certain embodiments, dissolution profiles of FEN from dried composite core-shell hydrogels;

FIG. 23 shows a determination of the lag time (t_L) from dissolution profiles, according to some embodiments;

FIG. 24A shows the lag time of some composite hydrogels, according to certain embodiments;

FIG. 24B shows, in accordance with some embodiments, the rate of dissolution of some composite hydrogels;

FIG. 25A shows dissolution profiles of fenofibrate from dried composite core-shell hydrogels before and after the removal of the PVA shell;

FIG. 25B shows an SEM image of a composite core-shell hydrogel containing FEN nanocrystals before PVA shell removal, according to certain embodiments; and

FIG. 25C shows an SEM image of a composite core-shell hydrogel containing FEN nanocrystals after PVA shell removal, according to certain embodiments.

### DETAILED DESCRIPTION

Compositions, methods, and systems for controlling crystallization of an agent (e.g., pharmaceutically active agent such as a hydrophobic drug) are generally described. In some embodiments, an agent is crystallized in the presence of polymer matrices, such as polymer particles. The polymer matrix may influence at least a portion of the crystallization process (e.g., nucleation or crystal growth) and/or the resulting composition (e.g., crystals). In some such embodiments, the polymer matrix allows one or more aspect of the process and/or composition to be controlled and/or altered. For instance, the polymer matrix may act as a crystallization promoter (e.g., heteronucleant) and/or acceptable carriers of the crystallized agent. In certain embodiments, the polymer matrix described herein, can be used with any agent regardless of its chemical and/or physical properties (e.g., solubility). Methods utilizing the polymer matrices may allow certain aspects of the resulting composition (e.g., crystal size, weight percentage of crystals) to be altered and/or controlled.

Crystallization of an agent within certain polymer matrices may be especially advantageous. For instance, as will be described in further detail below, crystallization of an agent within a matrix that has and/or forms a desirable structure (e.g., a composite core-shell particle) may be beneficial for certain applications, such as for the delivery of certain hydrophobic drugs. Accordingly, composite core-shell particles as well as related components and methods are also described herein. Composite core-shell particles typically comprise one or more first polymers (e.g., polymer(s) within a polymer matrix) in the core, and one or more second polymers (e.g., shell-forming polymer(s)) in the shell. Certain embodiments are related to composite core-shell particles where the shell comprises poly(vinyl alcohol) (PVA) (that is, in these embodiments the shell includes PVA and can include other components as well) and the formation of composite core-shell particles where the shell comprises PVA. It may be beneficial to form composite core-shell particles where the shell comprises PVA because PVA may form the shell during the performance of steps that would otherwise be performed (e.g., removing one or more solvents from an emulsion). Thus, it may be easier to form composite core-shell particles with PVA shells than with shells comprising other polymers. In some embodiments, the composite core-shell particles have a core which comprises a matrix (e.g., a cross-linked polymer) and one or more crystals (e.g., one or more crystals of a crystalline hydrophobic drug).

In many applications, certain polymer matrices (e.g., matrices comprising one or more first polymers) may be advantageous because they affect the crystallization process and/or resulting composition in a desirable manner. That is, in some cases it is advantageous to control and/or alter the crystallization process and/or the resulting composition. As used herein, references to the term “crystallization process” refer to the formation of crystalline solids from other states of matter which are not crystalline solids, such as liquid solutions and amorphous solids). Though most crystallization processes are heterogeneous and might be altered through the use of appropriately designed heteronucleant, the crystallization process, including nucleation behavior, is mostly unpredictable. The unpredictability of crystallization as well as the practical constraints on crystallization methods (e.g., in industrial practice) hamper the rational design of suitable materials capable of influencing crystallization. Therefore, conventional crystallization methods seek to control crystallization by adjusting parameters such as saturation level, temperature profile, solvent selection, stirring speed, etc. In general, the determination of appropriate crystallization parameters can be time-consuming and/or the parameters may have to be determined for each agent or class of agents.

It has been discovered, within the context of the present invention, that certain polymer matrices (e.g., matrices comprising one or more first polymer(s)) can control and/or alter the crystallization process (e.g., nucleation kinetics) and/or the resulting crystals (e.g., crystal size) without having to design a new polymer matrix for each agent or class of agents. In some embodiments, the polymer matrix is compatible with the intended use of the crystallized agent, such that at least a portion of the polymer matrix is not removed prior to use of the crystallized agent. In such cases, the need for post-crystallization processing relating to the polymer matrix is reduced or eliminated.

As described herein, an agent (e.g., a hydrophobic drug) may be crystallized in the presence of polymer matrices (e.g., matrices comprising one or more first polymer(s), hydrogel particles such as cross-linked hydrogel particles which optionally comprise a shell). In some embodiments, a method of crystallizing an agent involves associating the agent with a polymer matrix prior to crystallization and inducing crystallization of the agent while it is associated with the polymer matrix. For example, the agent may be at least partially encapsulated by a polymer matrix (e.g., polymer particle) prior to and during crystallization. In some embodiments, after crystallization, the polymer matrix may comprise crystals of the agent (e.g., the agent may form a dispersion in the polymer matrix). In some instances, the diameter of at least a portion of the crystals (e.g., average crystal diameter) may be greater than the pore size (i.e., mesh size) of the polymer matrix. In some such embodiments, at least a portion of the crystals are confined within and/or primarily encapsulated by the polymer matrix (e.g., polymer particle).

As noted above, the agent (e.g., hydrophobic drug) may be associated with the polymer matrices (e.g., matrices comprising one or more first polymer(s)) prior to crystallization. In some embodiments, the agent and the polymer matrices may be dissolved in a common solvent and allowed to associate. In other embodiments, the solubility of an agent, in at least one solvent is substantially different than the solubility of at least one precursor of the polymer matrix (e.g., monomers, polymer molecules) that affects the ability of the polymer matrix to be carried in the solvent without precipitating out. For instance, the agent may have a relatively low solubility (e.g., solubility of less than about 1 mg/ml) in a solvent (e.g., aqueous based solvent) and at least one precursor of the polymer matrix may have a relatively high solubility (e.g., greater than about 10 mg/ml) in the solvent (e.g. aqueous based solvent), such that the polymer matrix also has a relatively high solubility in the solvent (e.g., aqueous based solvent). In some such embodiments, an association between the agent and the polymer matrix, at adequate concentrations of the agent and matrix, cannot be readily formed through dissolution in a common solvent.

As used herein, solubility, and accordingly dissolution, with respect to the polymer matrix (e.g., a matrix comprising one or more first polymer(s)) may refer to the ability of the of the polymer matrix to be carried in the solvent without precipitating out. The solubility may be expressed in terms of concentration of the saturated solution of the polymer matrix at standard conditions.

In some embodiments, an emulsion system may be used to associate a polymer matrix (e.g., a matrix comprising one or more first polymer(s)) with an agent that has a substantially different solubility in at least one solvent than the polymer matrix or precursor (e.g., a hydrophobic drug). The agent may have a relatively low solubility in a first solvent in which the polymer matrix or precursor is dissolved. In some such embodiments, the agent is dissolved in a second solvent that is substantially immiscible with the first solvent. The polymer matrix or precursor in the first solvent may be combined with the agent in the second solvent to form an emulsion. In certain embodiments, the dispersed phase of the emulsion is the second solvent comprising the dissolved agent (i.e., the agent is present in droplets dispersed within the continuous phase, such as droplets of a non-aqueous phase dispersed within an aqueous phase) and the continuous phase is the first solvent comprising the polymer matrix or precursor. In other embodiments, the dispersed phase of the emulsion is the first solvent comprising the polymer matrix or precursor and the continuous phase is the second solvent comprising the dissolved agent. In some embodiments, the polymer matrix or precursor may be cross-linked or further cross-linked while in the emulsion (e.g., as the continuous phase). That is, the polymer matrix or precursor may be at least partially cross-linked while in the emulsion. In some embodiments, the polymer matrix or precursor may be cross-linked to a degree sufficient to form a gel. In some embodiments in which the continuous phase comprises the polymer matrix, at least a portion of the dispersed phase comprising the agent may be confined within and/or primarily encapsulated by the polymer matrix.

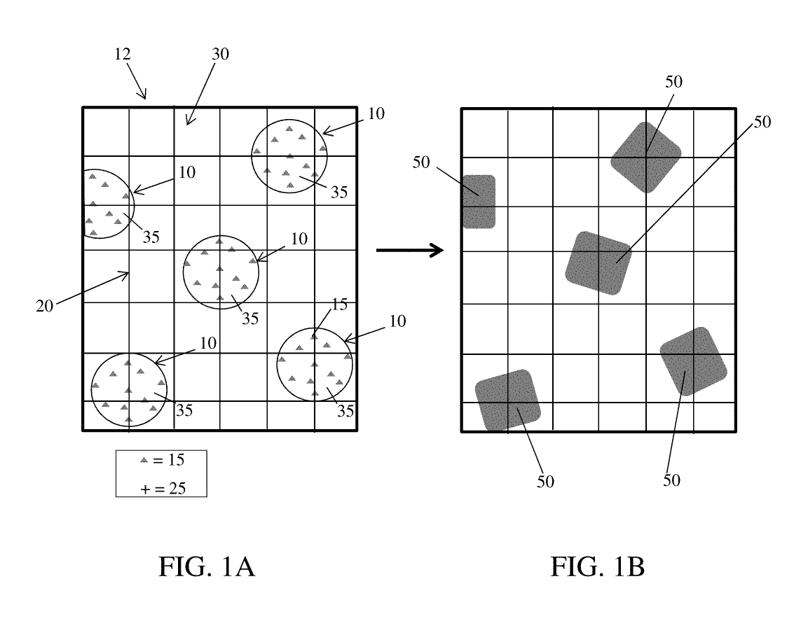

A non-limiting example of a method of crystallizing an agent (e.g., a hydrophobic drug) that has a substantially different solubility than the polymer matrix (e.g., a matrix comprising one or more first polymer(s)) in at least one solvent is shown in FIG. 1. FIG. 1 is a schematic illustration showing a cross-section of a portion of a single polymer matrix before and after crystallization, according to certain embodiments. As illustrated in FIG. 1, prior to crystallization, an emulsion is formed to associate the polymer matrix with the agent. Fluid droplets 10 comprising an agent 15 (e.g., pharmaceutically active agent) may be dispersed within a continuous phase 12 comprising the polymer matrix 20 having cross-links 25 and a first solvent 30 that is substantially immiscible with second solvent 35 used to form the fluid droplets. In some embodiments, the first solvent is a polar solvent, such as water (i.e., the first solvent may be an aqueous carrier). In other embodiments, the first solvent is an apolar and/or organic solvent (i.e., the first solvent may be a non-aqueous carrier). For example, when the first solvent is a polar solvent (e.g., water), the fluid in the droplet (i.e., second solvent) is an apolar solvent. In another example, when the first solvent is an apolar solvent, the second solvent is a polar solvent. In some embodiments, the mesh size of the polymer matrix relative to the droplet size may cause at least a portion of the fluid droplets to be confined and retained in the polymer matrix.

As described above, certain inventive articles and methods may relate to compositions which comprise aqueous and/or non-aqueous carriers. As used herein, non-aqueous carriers are species which do not comprise water or which comprise less than 5 wt % water; aqueous carriers are species which comprise water in an amount greater than 5 wt %. The non-aqueous carrier may be any suitable fluid that is not water. In some embodiments, the non-aqueous carrier may be a fluid that is not miscible with water. In some embodiments, the non-aqueous carrier may be an organic solvent.

After the polymer matrix (e.g., a matrix comprising one or more first polymer(s)) is associated with the agent (e.g., hydrophobic drug), the agent in the fluid droplet may be crystallized within the polymer matrix (e.g. within a polymer particle) by any suitable method known to those of ordinary skill in the art to induce crystallization (e.g., evaporation, temperature shock, chemical interference). In some embodiments, an agent such as a hydrophobic drug may be crystallized by removing of sufficient amounts of one more solvents (e.g., a first solvent, a second solvent, an aqueous carrier, a non-aqueous carrier) from the polymer matrix. Without being bound by theory, it is believed that the fluid droplets within the polymer matrix serve as compartmentalized units where crystallization can be achieved. It is believed that since these compartmentalized units are accessible and retained within the polymer matrix, crystallization can be induced.

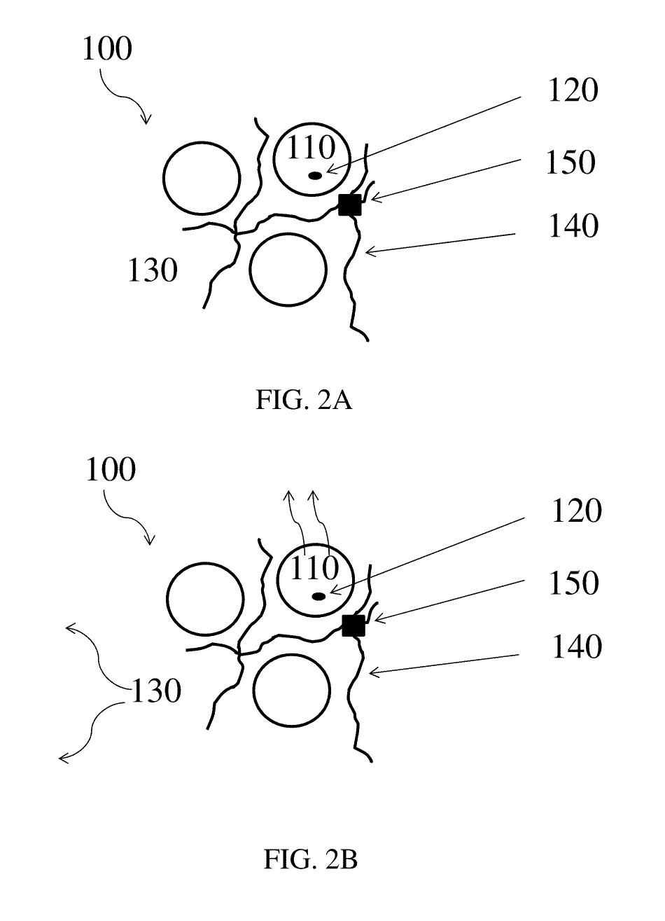

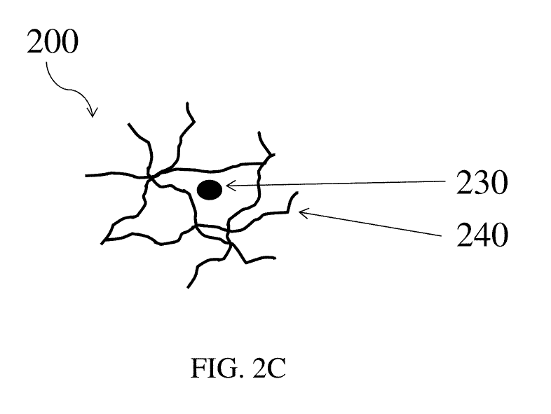

One suitable method for crystallizing an agent within a polymer matrix (e.g., a matrix comprising one or more first polymer(s)) may comprise forming a cross-linked emulsion comprising a second solvent (e.g., a non-aqueous carrier; typically containing an agent such as a hydrophobic drug) dispersed in a first solvent (e.g., an aqueous carrier) and then drying the emulsion (e.g., removing sufficient amounts one or more solvents from the emulsion) to form a dispersion of the agent (e.g., solid hydrophobic drug) in a cross-linked matrix. During the drying process, both the first and second solvents may be removed. When the removal of these species occurs over overlapping periods of time, the agent may solidify to form crystals with desirable properties, such as crystals with an average cross-sectional diameter of less than or equal to 1 micron. A non-limiting example of a method (e.g., a method for forming composite core-shell particles), according to some embodiments, is shown in FIG. 2A, FIG. 2B, and FIG. 2C. In these FIGs., the method comprises forming an emulsion that is at least partially cross-linked from a first solvent that is an aqueous carrier and a second solvent that is non-aqueous carrier comprising an agent that is a hydrophobic drug. However, this method may be applied more generally to other combinations of first solvents and second solvents and to compositions comprising agents that are not hydrophobic drugs. Without wishing to be bound by theory, hydrophobic species such as hydrophobic drugs are typically considered to be species with a water solubility of less than or equal to 5 wt %. Drugs are a class of active pharmaceutical agents typically considered to be therapeutic or medicinal agents.

As shown in FIG. 2A, an exemplary method may comprise forming a cross-linked emulsion 100 comprising a non-aqueous carrier 110 with dissolved hydrophobic drug 120 dispersed in an aqueous carrier 130. The emulsion may be cross-linked to a degree sufficient to form a gel, such as a hydrogel. Aqueous carrier 130 comprises first polymer 140 which is cross-linked by cross-linking agent 150. In some embodiments, the hydrophobic drug is dissolved in the non-aqueous phase (e.g., dissolved in a non-aqueous carrier). It should be noted that in some embodiments, the at least partially cross-linked emulsion is simultaneously formed and cross-linked, while in other embodiments the cross-linked emulsion is first formed and then cross-linked. The emulsion may be formed by adding the dispersed phase to the continuous phase. This may be accomplished by, e.g., dripping the dispersed phase into the continuous phase while subjecting the continuous phase to stirring.

In certain embodiments, emulsion formation may followed by additional treatments to further reduce droplet size, such as by using ultrasonication. In some embodiments, the droplet size may be reduced to a level such that a nanoemulsion is formed.

After emulsion formation and cross-linking, the method may then comprise removing the aqueous carrier and the non-aqueous carrier, thereby crystallizing the drug as a dispersion of solid hydrophobic drug in a cross-linked matrix comprising the first polymer. This step is shown in FIG. 2B, where aqueous carrier 130 and non-aqueous carrier 110 are removed to form article 200 shown in FIG. 2C comprising crystalline solid hydrophobic drug 230 dispersed in cross-linked polymer matrix 240. In some, but not necessarily all, embodiments, the cross-linked polymer matrix may be a composite core-shell particle or a component thereof (e.g., a core thereof), as described further elsewhere herein.

As described above, certain inventive methods relate to cross-linking emulsions (i.e., to forming chemical bonds between distinct polymer chains within the emulsions) and/or cross-linking emulsions to a degree sufficient to form a gel. In certain embodiments, an emulsion may be formed and cross-linked essentially simultaneously. As used herein, essentially simultaneously forming and cross-linking an emulsion comprises performing a single step such that the emulsion is both formed and at least partially cross-linked over a period of not greater than 60 minutes in a single vessel. This may be achieved by, for example, simultaneously introducing a non-aqueous carrier into an aqueous carrier containing a cross-linking agent and cross-linking the cross-linking agent. In some embodiments, the formation of the emulsion and its cross-linking may occur in two steps. The first step may be forming the emulsion by introducing the non-aqueous carrier into the aqueous carrier, which may be followed by a second step of cross-linking the cross-linkable agent. References below to methods for cross-linking emulsions should be understood to encompass both cross-linking that occurs simultaneously with emulsion formation and cross-linking that occurs after the emulsion has formed.

As described above, certain inventive methods relate to removing one or more solvents (e.g., aqueous and/or non-aqueous carriers) from emulsions (e.g., cross-linked emulsions). Removing an aqueous or non-aqueous carrier from an emulsion may comprise performing a step such less than 5% of the aqueous carrier or non-aqueous carrier present prior to the removing step is present at the conclusion of the removing step. In certain embodiments, two solvents present in an emulsion (e.g., an aqueous carrier and a non-aqueous carrier) may be removed essentially simultaneously. As used herein, essentially simultaneously removing two or more solvents from an emulsion comprises performing a single step such that the aqueous carrier and the non-aqueous carrier are removed at substantially similar rates.

In some embodiments, one or more solvents (e.g., an aqueous carrier, a non-aqueous carrier) may be removed from an emulsion (e.g., a nanoemulsion, cross-linked emulsion, an emulsion that is a gel) by heating the emulsion. In certain embodiments, removing one or more solvents (e.g., essentially simultaneously, not essentially simultaneously) may comprise heating the emulsion to a temperature of at least 30° C., at least 35° C., at least 40° C., at least 45° C., at least 50° C., at least 55° C., at least 60° C., at least 65° C., at least 70° C., or at least 75° C. In certain embodiments, removing the aqueous and non-aqueous carriers may comprise heating the emulsion to a temperature of less than 80° C., less than 75° C., less than 70° C., less than 65° C., less than 60° C., less than 55° C., less than 50° C., less than 45° C., less than 40° C., or less than 35° C. Combinations of the above-referenced ranges are also possible (e.g., greater than or equal to 30° C. and less than or equal to 80° C., or greater than or equal to 60° C. and less than or equal to 80° C.). Other ranges are also possible. It should also be noted that the emulsion may be heated at either ambient pressure or under reduced pressure.

In some embodiments, as illustrated in FIG. 1B, a single crystal 50 is formed in the fluid droplet (e.g., a fluid droplet in an emulsion, a fluid droplet in a nanoemulsion, a fluid droplet in a cross-linked emulsion, a fluid droplet in a polymer matrix, a fluid droplet in a matrix comprising one or more first polymer(s)). For instance, a polymer particle comprising twenty fluid droplets has no more than twenty crystals (e.g., crystals of the agent, crystals of a hydrophobic drug) after crystallization. In some embodiments, the resulting crystals are retained within or associated with the polymer matrix (e.g., a matrix comprising one or more first polymer(s)). In some such cases, the average diameter of the crystals may be greater than the average mesh size of the polymer matrix. In certain embodiments, the geometry of the crystals may be controlled, in part, by the geometry of fluid droplets. For instance, the geometry of the fluid droplets may influence crystal diameter. Without wishing to be bound by theory, it is believed that the diameter of the fluid droplet sets an upper limit for the crystal diameter and the coefficient of variation in the crystal diameter. That is, the maximum average diameter and coefficient of variation is substantially the same as that of the fluid droplets. It is also believed that the diameter of the crystal is also a function of the concentration of the agent in the dispersed phase, such that the crystal diameter decreases as the concentration of the agent decreases and the crystal size is substantially the same as the droplet size when the fluid in the droplet is saturated with the agent.

In some embodiments, the ratio of the crystal diameter (e.g., the crystal diameter of crystals of the agent, the crystal diameter of crystals of a hydrophobic drug) to the diameter of the fluid droplet (e.g., the diameter of a fluid droplet in an emulsion, the diameter of a fluid droplet in a nanoemulsion, the diameter of a fluid droplet in a cross-linked emulsion, the diameter of a fluid droplet in an emulsion that is a gel) is less than or equal to about 1:1, less than or equal to about 0.75:1, less than or equal to about 0.5:1, less than or equal to about 0.25:1, less than or equal to about 0.10:1, or less than or equal to about 0.05:1.

In some embodiments, the average diameter of the crystals formed using an emulsion system (e.g., formed using a nanoemulsion system, formed using a cross-linked emulsion system, formed using an emulsion system that is a gel) may be relatively small (e.g., the crystals may be in the form of a nanodispersion in the polymeric matrix). In some cases, the average diameter of the crystals may be is less than or equal to about 100 microns, less than or equal to about 10 microns, less than or equal to about 1 micron, less than or equal to about 0.8 microns, less than or equal to about 0.7 microns, less than or equal to about 0.65 microns, less than or equal to about 0.6 microns, less than or equal to about 0.55 microns, less than or equal to about 0.5 microns, less than or equal to about 0.45 microns, less than or equal to about 0.4 microns, less than or equal to about 0.35 microns, less than or equal to about 0.3 microns, less than or equal to about 0.2 microns, less than or equal to about 0.1 microns, less than or equal to about 0.08 microns less than or equal to about 0.05 microns, or less than or equal to about 0.02 microns. In some embodiments, the average diameter of the crystals formed is greater than or equal to about 0.01 microns, greater than or equal to about 0.02 microns, greater than or equal to about 0.05 microns, greater than or equal to about 0.08 microns, greater than or equal to about 0.1 microns, greater than or equal to about 0.2 microns, greater than or equal to about 0.3 microns, greater than or equal to about 0.35 microns, greater than or equal to about 0.4 microns, greater than or equal to about 0.45 microns, greater than or equal to about 0.5 microns, greater than or equal to about 0.55 microns, greater than or equal to about 0.6 microns, greater than or equal to about 0.65 microns, greater than or equal to about 0.7 microns, greater than or equal to about 0.8 microns, greater than or equal to about 1 micron, or greater than or equal to about 10 microns. In some instances, the average diameter of the crystals formed using an emulsion system (e.g., a nanoemulsion system, a cross-linked emulsion system, an emulsion system that is a gel) may be between about 0.01 microns and about 100 microns, between about 0.01 microns and about 10 microns, between about 0.01 microns and about 1 microns, between about 0.45 microns and about 0.65 microns, between about 0.1 microns and 0.65 microns, or between about 0.01 microns and about 0.4 microns.

In some embodiments, the coefficient of variation in the average crystal diameter (e.g., the average crystal diameter of crystals of the agent, the average crystal diameter of crystals of a hydrophobic drug) is less than or equal to about 20%, less than or equal to about 15%, less than or equal to about 10% or less than or equal to about 5%.

As shown in FIG. 1, an emulsion (e.g., a nanoemulsion, a cross-linked emulsion, an emulsion that is a gel) may be used to associate agents (e.g., hydrophobic drugs) and polymer matrices (e.g., matrices comprising one or more first polymer(s)) with a substantially different solubility in at least one solvent (e.g., water). Without being bound by theory, it is believed that solubility of the agent and the polymer matrices in the emulsion solvents is an important factor in the formation of a suitable emulsion system. It is believed that the agent should be relatively insoluble in the first solvent (e.g., a continuous phase, a phase comprising an aqueous carrier) and highly soluble in the second solvent (e.g., a dispersed phase, a phase comprising a non-aqueous carrier). Conversely, it is believed that the polymer matrix should be relatively insoluble in the second solvent and highly soluble in the first solvent (e.g., continuous phase). For instance, the solubility of the agent in the first solvent and the solubility of the polymer matrix and/or precursor in the second solvent is less than about 0.25 mg/ml, less than about 0.1 mg/ml, less than about 0.05 mg/ml, less than about 0.01 mg/ml, less than about 0.005 mg/ml, or less than about 0.001 mg/ml.

The solubility of the agent (e.g., a hydrophobic drug) in the second solvent (e.g., a non-aqueous carrier) and the solubility of the polymer matrix (e.g., a matrix comprising one or more first polymer(s)) and/or precursor in the first solvent (e.g., an aqueous carrier) may be greater than or equal to about 0.01 g/ml, greater than or equal to about 0.05 g/ml, greater than or equal to about 0.1 g/ml, greater than or equal to about 0.5 g/ml, greater than or equal to about 1.0 g/ml, greater than or equal to about 5 g/ml, greater than or equal to about 10 g/ml, greater than or equal to about 25 g/ml, greater than or equal to about 50 g/ml, or greater than or equal to about 75 g/ml. In some embodiments, the solubility of the agent in the second solvent and the solubility of the polymer matrix and/or precursor in the first solvent may be between about 0.01 g/ml and about 100 g/ml, between about 0.1 g/ml and about 100 g/ml, between about 1.0 g/mL and about 100 g/mL, or between about 10 g/ml and about 100 g/ml.

In some embodiments, the solubility of a precursor of the polymer matrix and/or the polymer matrix (e.g., a matrix comprising one or more first polymer(s)) in the first solvent (e.g., an aqueous carrier) may be at least 2 times, at least 5 times, at least 10 times, at least 25 times, at least 50 times, at least 75 times, at least 100 times, at least 150 times, at least 200 times, at least 250 times, at least 300 times, at least 350 times, at least 400 times, at least 450 times, or at least 500 times greater than the solubility of the agent (e.g., a hydrophobic drug) in the first solvent. For example, when the first solvent is water, the solubility of a hydrophilic precursor of the polymer matrix (e.g., a first polymer molecule) is at least 100 times (e.g. at least 200 times, at least 300 times, at least 400 times, at least 500 times) greater than a hydrophobic agent (e.g., a pharmaceutically active agent, a hydrophobic drug).

In general, any suitable solvents may be used as a first and a second solvent provided that the first and second solvents are substantially immiscible and the agent (e.g., a hydrophobic drug) and the polymer matrix (e.g., a matrix comprising one or more first polymer(s)) has the requisite solubility in each solvent. For instance, in some embodiments, the solvent(s) may be selected from FDA approved solvents (e.g., FDA generally regarded as safe (GRAS) solvents, FDA approved class III solvents). Non-limiting examples of suitable polar solvents include water, aqueous based solvents, acetic acid, acetone, dimethylformamide, acetonitrile, ethyl formate, formic acid, dimethyl sulfoxide, dichloromethane, butanol, 3-methyl-2-butanol, ethanol, methanol, pentanol, acetic acid, isopropanol, propanol, 2-methyl-1-propanol, nitromethane, and/or combinations thereof. Non-limiting examples of suitable apolar solvents include ethyl acetate, isobutyl acetate, methyl acetate, propyl acetate, methyl pentane, methylethylketone, methylisobutylketone, isobutyl ketone, cumene, tert-butyl-methylester ether, heptane, hexane, anisole, toluene, chloroform, diethyl ether, benzene, isopropyl acetate, cyclohexane, ether (e.g., ethyl ether), dioxane, tetrahydrofuran, FDA GRAS oils (e.g., corn oil, olive oil) and/or combinations thereof. Those of ordinary skill in the art can select suitable substantially immiscible fluids, using contact angle measurements or the like, based on the description herein. Organic solvents that are FDA approved may be preferred for use as a first solvent, second solvent, and/or non-aqueous carrier in some embodiments.

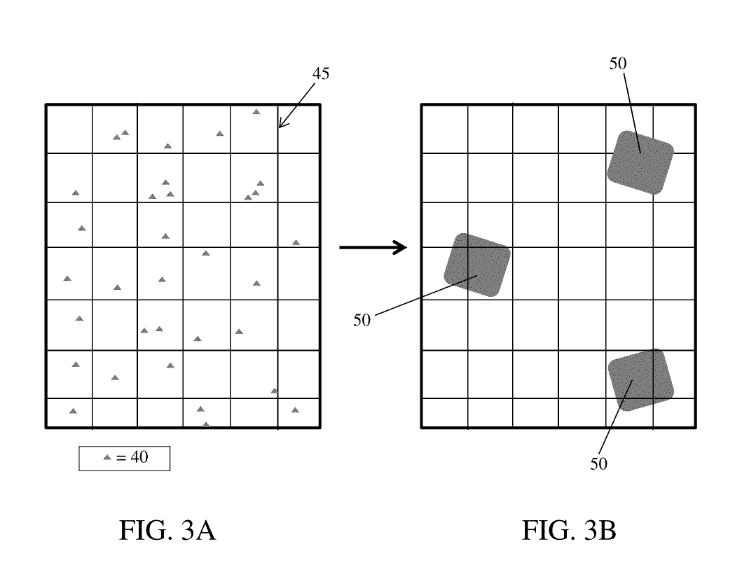

As described herein, the agent (e.g., a hydrophobic drug) and the polymer matrix (e.g., a matrix comprising one or more first polymer(s)) may have similar solubility in at least one solvent (e.g., water, an aqueous carrier, a non-aqueous carrier), such that the agent and the polymer matrices can be readily associated at adequate concentrations. In general, any suitable method known to those of skill in the art may be used to cause the agent and polymer matrices to associate. In some instances, the agent and the polymer matrices are incubated in a common solvent and allowed to form selective interactions. In some cases, a precursor of the polymer matrix and the agent are incubated in the common solvent and allowed to form selective interactions. The polymer matrix precursor may be cross-linked after association with the agent to form a plurality of polymer matrices. An exemplary method of crystallizing an agent and polymer matrix in a common solvent is illustrated in FIG. 3. FIG. 3 is a schematic illustration showing a cross-section of a portion of the polymer matrix before and after polymerization. As illustrated in FIG. 3A, prior to crystallization, the agent 40 may be associated with the polymer matrix 45 via selective interaction(s). The agent may be crystallized in the presence of the polymer matrix. As illustrated in FIG. 3B, the resulting crystals 50 may be associated with the polymer matrix. In some embodiments, the crystals may be primarily encapsulated or confined within the matrix.

In some embodiments, the average diameter of the crystals (e.g., the average diameter of crystals of an agent, the average diameter of crystals of a hydrophobic drug) formed using a common solvent is less than or equal to about 600 microns, less than or equal to about 500 microns, less than or equal to about 400 microns, less than or equal to about 300 microns, less than or equal to about 200 microns, less than or equal to about 100 microns, less than or equal to about 50 microns, less than or equal to about 20 microns, less than or equal to about 10 microns, less than or equal to about 1 micron, less than or equal to about 0.8 microns, less than or equal to about 0.6 microns, less than or equal to about 0.4 microns, less than or equal to about 0.2 microns, less than or equal to about 0.1 microns, less than or equal to about 0.05 microns, or less than or equal to about 0.02 microns. In some embodiments, the average diameter of the crystals formed using a common solvent is between about 0.01 microns and about 600 microns, between about 0.02 microns and about 600 microns, between about 0.05 microns and about 600 microns, between about 0.1 microns and about 600 microns, between about 0.01 microns and about 100 microns, between about 0.01 microns and about 10 microns, or between about 0.01 microns and about 1 micron. In some cases, a nanodispersion of crystals may be formed by using a common solvent.

In some embodiments, the coefficient of variation in the average crystal diameter is less than or equal to about 20%, less than or equal to about 15%, less than or equal to about 10% or less than or equal to about 5%.

As described herein, the polymer matrices (e.g., matrices comprising one or more first polymer) may serve as a crystallization promoter (e.g., of an agent, of a hydrophobic drug). Without being bound by theory, it is believed that the average nucleation induction time of agent is influenced by the mesh size of the polymer matrix. It is believed that there is an optimal range in mesh size that allows sufficient favorable interaction between the polymer matrix and the agent molecules to occur. It is also believed that optimal range in mesh size allows for agent molecules associated with the polymer molecules in the polymer matrix to come within sufficient proximity to form a nucleus of agent molecules and polymer molecules. For relatively small mesh sizes, an agent molecule may “see” more polymer chains than solvent molecules, which enhances the interaction between the agent molecule and the polymer molecules in the polymer matrix. For relatively large mesh sizes, it is believed that the agent molecules and the polymer molecules in the matrix are separated from each other, such that the interaction between the agent molecules and the polymer molecules is not enhanced.

In some embodiments, crystallizing an agent (e.g., a hydrophobic drug) in the presence of a polymer matrix (e.g., a matrix comprising one or more first polymer(s)) decreases the average nucleation induction time compared to crystallizing an agent in the absence of the polymer matrix under identical crystallization conditions. For instance, in some embodiments, the percent decrease in the average nucleation induction time may be greater than or equal to about 10%, greater than or equal to about 20%, greater than or equal to about 40%, greater than or equal to about 60%, greater than or equal to about 80%, greater than or equal to about 100%, or greater than or equal to about 150%. In some instances, the percent decrease may be less than or equal to about 200%, less than or equal to about 150%, less than or equal to about 100%, less than or equal to about 80%, less than or equal to about 60%, less than or equal to about 40%, less than or equal to about 20%, less than or equal to about 10%, or less than or equal to about 5%. All combinations of the above-referenced ranges are also possible (e.g., greater than or equal to about 10% and less than or equal to about 200%, greater than or equal to about 10% and less than or equal to about 100%). Crystal nucleation was determined by continuous monitoring of the sample using an inverted microscope. The onset of crystallization was the point at which the first crystal appeared. Statistical analysis methods known to those of ordinary skill in the art were used to calculate the average induction time.

Without being bound by theory, it is believed that the average nucleation induction time of an agent (e.g., a hydrophobic drug) is influenced by the mesh size (i.e., pore size) of the polymer matrix (e.g., a matrix comprising one or more first polymer(s)). In some embodiments, the mesh size of the polymer matrix may be less than or equal to about 20 nm, less than or equal to about 15 nm, less than or equal to about 12 nm, less than or equal to about 10 nm, or less than or equal to about 8 nm. In some embodiments, the mesh size of the polymer is less than or equal to about 10 nm. Mesh size may be determined via oscillatory rheology using frequency sweep measurements at a fixed strain modeled in terms of the generalized Maxwell model.

In some embodiments, the average mesh size of a polymer matrix (e.g., a matrix comprising one or more first polymer(s)) may be less than the average diameter of the fluid droplets in the dispersed phase of the emulsion and/or the crystal size of the crystallized agent (e.g., a hydrophobic drug). For instance, in some embodiments, the ratio of the average mesh size to the average diameter of the fluid droplets and/or the crystals is less than or equal to about 0.95:1, less than or equal to about 0.8:1, less than or equal to about 0.6:1, less than or equal to about 0.4:1, less than or equal to about 0.2:1, or less than or equal to about 0.1:1.

In some embodiments, the average diameter of the fluid droplets (e.g., in an emulsion, in a nanoemulsion, in a cross-linked emulsion, in an emulsion that is a gel) is less than or equal to about 100 microns, less than or equal to about 10 microns, less than or equal to about 1 micron, less than or equal to about 0.8 microns, less than or equal to about 0.7 microns, less than or equal to about 0.65 microns, less than or equal to about 0.6 microns, less than or equal to about 0.55 microns, less than or equal to about 0.5 microns, less than or equal to about 0.45 microns, less than or equal to about 0.4 microns, less than or equal to about 0.35 microns, less than or equal to about 0.3 microns, less than or equal to about 0.2 microns, less than or equal to about 0.1 microns, less than or equal to about 0.05 microns, less than or equal to about 0.02 microns, or less than or equal to about 0.01 microns. In some embodiments, the average diameter of the fluid droplets is greater than or equal to about 0.005 microns, greater than or equal to about 0.01 microns, greater than or equal to about 0.02 microns, greater than or equal to about 0.05 microns, greater than or equal to about 0.1 microns, greater than or equal to about 0.2 microns, greater than or equal to about 0.3 microns, greater than or equal to about 0.35 microns, greater than or equal to about 0.4 microns, greater than or equal to about 0.45 microns, greater than or equal to about 0.5 microns, greater than or equal to about 0.55 microns, greater than or equal to about 0.6 microns, greater than or equal to about 0.65 microns, greater than or equal to about 0.7 microns, greater than or equal to about 0.7 microns, greater than or equal to about 0.8 microns, greater than or equal to about 1 micron, or greater than or equal to about 10 microns. Combinations of the above-referenced ranges are also possible. In some instances, the average diameter of the fluid droplets is between about 0.01 microns and about 100 microns, between about 0.01 microns and about 10 microns, between about 0.01 microns and about 1 micron, between about 0.45 microns and 0.65 microns, between about 0.1 microns and about 0.65 microns, or less than or between about 0.01 microns and about 0.4 microns. Other ranges are also possible. Average droplet cross-sectional diameter can be measured by diluting the emulsion or nanoemulsion in deionized water and then performing dynamic light scattering (DLS) at a scattering angle of 90° and a temperature of 25° C. using a Wyatt Technology DynaPro NanoStar Instrument. Average cross-sectional diameter and polydispersity may be extracted from raw DLS data using second order cumulant analysis.

In some embodiments, the coefficient of variation in the average droplet diameter (e.g., in an emulsion, in a nanoemulsion, in a cross-linked emulsion, in an emulsion that is a gel) is less than or equal to about 20%, less than or equal to about 15%, less than or equal to about 10% or less than or equal to about 5%.

When present, an emulsion (e.g., a nanoemulsion, a cross-linked emulsion, an emulsion that is a gel) may have any suitable volume fraction of the dispersed phase. In some embodiments, the emulsion has a dispersed phase volume fraction of greater than or equal to 5%, greater than or equal to 10%, greater than or equal to 15%, greater than or equal to 20%, greater than or equal to 25%, greater than or equal to 30%, or greater than or equal to 35%. In certain embodiments, the emulsion has a dispersed phase volume fraction of less than or equal to 40%, less than or equal to 35%, less than or equal to 30%, less than or equal to 25%, less than or equal to 20%, less than or equal to 15%, or less than or equal to 10%. Combinations of the above-referenced ranges are also possible (e.g., greater than or equal to 5% and less than or equal to 40%). Other ranges are also possible. The volume fraction of the dispersed phase may be determined by dividing the added dispersed phase volume by the sum of the continuous phase volume and the added dispersed phase volume and multiplying by 100%.

In some embodiments, as described elsewhere herein, an emulsion (e.g., a cross-linked emulsion, a nanoemulsion, an emulsion that is a gel) may further comprise one or more second polymer(s) (e.g., one or more shell-forming polymer(s)) in addition to polymer matrix (e.g., one or more first polymer(s)). The second polymer(s) may be polymer(s) for which less than or equal to 5% of the monomers of each polymer chain are cross-linked to another polymer chain. As used herein, polymer chains are chains of polymerized monomer units; polymer chains may be linear chains, branched chains, dendritic chains, or other chains that alone do not comprise a cross-linked polymer network. Cross-links refer to chemical bonds (such as covalent bonds) between two or more distinct polymer chains.

In some embodiments, second polymer(s) (e.g., shell-forming polymer(s)) may be polymer(s) that have an affinity for the surfaces of emulsion droplets, such as a surfactant. A second polymer may be located at the surfaces of the emulsion droplets prior to drying, but may diffuse through the cross-linked emulsion as it is drying such that at least a portion of the second polymer is in a different location at the conclusion of the drying process. For example, in certain embodiments, the emulsion may form particles during the drying process and at least a portion of a second polymer may be located at the surface of the particles at the conclusion of the drying process. In some embodiments, the second polymer is poly(vinyl alcohol).

Methods and systems described herein may allow one or more property of the resulting composition to be controlled. For example, crystallizing an agent (e.g., a hydrophobic drug) in the presence of polymer matrices (e.g., matrices comprising one or more first polymer(s)) may allow the crystals to be associated with the polymer matrices after crystallization (e.g., as a dispersion in the polymer matrices). In certain embodiments, the majority of the crystals associated with a polymer matrix may be encapsulated within the polymer matrix. For instance, in some embodiments, the percentage of crystals that are encapsulated by the polymer matrix is greater than or equal to about 60%, greater than or equal to about 70%, greater than or equal to about 80%, greater than or equal to about 90%, greater than or equal to about 95%, greater than or equal to about 98%, or greater than or equal to about 99%. The percentage of crystals that are encapsulated by the polymer matrix may be determined using transmission electron microscopy.

In some embodiments, the polymer matrix (e.g., a matrix comprising one or more first polymer(s)) may serve as carriers for the crystals (e.g., crystals of an agent, crystals of a hydrophobic drug). For instance, a pharmaceutically active agent may be crystallized in the presence of a polymer particles, such that the pharmaceutically active agent is primarily encapsulated (e.g., greater than or equal to about 80%, greater than or equal to about 90%, greater than or equal to about 95%, greater than or equal to about 98%, or greater than or equal to about 99% encapsulated) in the polymer particles. The polymer particles may serve as particulate carriers for the pharmaceutically active agent.

In some embodiments, association via emulsion may allow the weight percentage of the agent (e.g., a hydrophobic drug) and the polymer matrix (e.g., a matrix comprising one or more first polymer(s)) in the composition to be controlled. For instance, the weight percentage of the agent may be controlled by varying the concentration of the dispersed phase in the emulsion and/or the concentration of the agent in the dispersed phase. In some embodiments, the emulsion may be formulated such that the resulting composition comprising crystals and polymer matrices has a relatively high weight percentage of crystals (e.g., greater than equal to about 75%). In general, the emulsion may be formulated to produce a composition with any suitable weight percentage of the crystallized agent. For instance, in some embodiments, the weight percentage of crystallized agent is greater than or equal to about 10%, greater than or equal to about 20%, greater than or equal to about 30%, greater than or equal to about 40%, greater than or equal to about 50%, greater than or equal to about 60%, greater than or equal to about 70%, or greater than or equal to about 80%. In some instances, the weight percentage of the crystallized agent is less than or equal to about 90%, less than or equal to about 80%, less than or equal to about 70%, less than or equal to about 60%, less than or equal to about 50%, less than or equal to about 40%, less than or equal to about 30%, or less than or equal to about 20%. All combinations of the above-referenced ranges are also possible (e.g., greater than or equal to about 20% and less than or equal to about 90%, or greater than or equal to about 40% and less than or equal to about 90%). Weight percentage, as used herein, refers to the dry weight percentage of the crystals (e.g., crystals of an agent such as a hydrophobic drug) within the composition (e.g., a composite core-shell particle comprising a core and a shell).

In some embodiments, association via non-emulsion methods, such as dissolution in a common solvent, may allow the weight percentage of the agent (e.g., a hydrophobic drug) and the polymer matrix (e.g., a matrix comprising one or more first polymer(s)) in the composition to be controlled. In general, the composition comprising the polymer matrix and the crystallized agent may have any suitable weight percentage of the crystallized agent. For instance, in some embodiments, the weight percentage of crystallized agent is greater than or equal to about 5%, greater than or equal to about 10%, greater than or equal to about 15%, greater than or equal to about 20%, greater than or equal to about 30%, or greater than or equal to about 40%. In some instances, the weight percentage of the crystallized agent is less than or equal to about 50%, less than or equal to about 40%, less than or equal to about 30%, or less than or equal to about 20%. All combinations of the above-referenced ranges are also possible (e.g., greater than or equal to about 5% and less than or equal to about 30%.

In some embodiments, the polymer matrix (e.g., a matrix comprising one or more first polymer(s)) may be designed to function as a suitable carrier for diverse application (e.g., methods involving the crystallized agent, products containing the crystallized agent). For instance, the polymer matrix may be designed to be, e.g., biocompatible so that it can be used in pharmaceutical compositions and/or consumer products. In some embodiments, the polymer matrix may comprise polymer molecules associated via chemical (e.g., covalent, non-covalent), physical (e.g., entanglement), and/or biological (e.g., receptor-ligand) interactions. In some embodiments, at least a portion of the interactions may form cross-links. In general, the polymer molecules may be cross-linked via any suitable interaction.

In some embodiments, the polymer molecules (e.g., first polymer molecules) may be associated via a chemical interaction, such as a chemical bond. The chemical bond may be a covalent bond or non-covalent bond. In some cases, the chemical bond is a non-covalent bond such as a hydrogen bond, ionic bond, dative bond, and/or a Van der Waals interaction. One or more of the polymer molecules may comprise functional groups capable of forming such bonds. It should be understood that covalent and non-covalent bonds between components may be formed by any type of reactions, as known to those of ordinary skill in the art, using the appropriate functional groups to undergo such reactions. Chemical interactions suitable for use with various embodiments described herein can be selected readily by those of ordinary skill in the art, based upon the description herein.

In some embodiments, the polymer molecules (e.g., first polymer molecules) may be associated via physical interactions. For example, in some embodiments, at least a portion of the polymer molecules are physically entangled.

In some embodiments, the polymer molecules (e.g., first polymer molecules) may be associated via biological interactions, such as a biological binding event (i.e., between complementary pairs of biological molecules). One or more of the polymer molecules may comprise biological molecules capable of forming such bonds. Examples of biological molecules that may form biological bonds between pairs of biological molecules include, but are not limited to, proteins, nucleic acids, glycoproteins, carbohydrates, hormones, and the like. Non-limiting examples include, but are not limited to, a protein/substrate pair, a nucleic acid/nucleic acid pair, a protein/nucleic acid pair, a peptide/peptide pair, a protein/protein pair, a small molecule/protein pair, a receptor/hormone pair, a biotin/avidin pair, a biotin/streptavidin pair, a drug/target pair, small molecule/peptide pair, a small molecule/protein pair, and/or combinations thereof. Biological interactions between polymer molecules for use in the embodiments described herein can be selected readily, by those of ordinary skill in the art, based upon the description herein as their function, examples of such biological interactions, and knowledge herein and in the art as to simple techniques for identifying suitable biological interactions.

It should be understood that the polymer matrix may be formed from more than one type of first polymer molecule and may have any suitable shape (e.g., particle, planar, non-planar) or dimension. For example, in some embodiments, the polymer matrices may be in particulate form. In some such embodiments, the particles may have an average diameter of greater than or equal to about 0.1 microns, greater than or equal to about 1 micron, greater than or equal to about 10 micron, greater than or equal to about 50 micron, greater than or equal to about 100 micron, greater than or equal to about 200 micron, greater than or equal to about 400 micron, greater than or equal to about 600 micron, greater than or equal to about 800 micron, or greater than or equal to about 1000 micron. In some instances, the average diameter of the polymer particles is less than or equal to about 3,000 microns, less than or equal to about 2,000 microns, less than or equal to about 1,000 microns, less than or equal to about 800 microns, less than or equal to about 600 microns, less than or equal to about 400 microns, less than or equal to about 200 microns, less than or equal to about 100 microns, less than or equal to about 50 microns, less than or equal to about 10 microns, or less than or equal to about 1 micron. All combinations of the above-referenced ranges are also possible (e.g., greater than or equal to about 1 micron and less than or equal to about 1,000 microns, greater than or equal to about 1 micron and less than or equal to about 600 microns, greater than or equal to about 0.1 microns and less than or equal to about 600 microns).

In some embodiments, at least a portion of the crystal (e.g., crystal of an agent, crystal of a hydrophobic drug) encapsulated within the polymer matrix has a portion that protrudes outside of the polymer matrix. In some such embodiments, a polymer shell can be formed around the polymer matrix to cover and mechanically protect the exposed portions of the crystals. In some embodiments, the polymer shell may be formed from the same or different polymer molecules as the polymer matrix. In certain cases, as also described elsewhere herein, the polymer shell may comprise PVA. In some embodiments, the polymer shell may also comprise an agent (e.g., a hydrophobic drug). In some instances, the agent in the shell may be any agent described herein with respect to the polymer matrices. In other embodiments, the polymer shell does not comprise an agent. In some instances, the polymer shell may be cross-linked. In other instances, the polymer shell may lack cross-links.

Composite core-shell particles, such as those that may be formed by methods described herein, may be particularly advantageous for drug delivery applications. If such particles are exposed to aqueous or physiological environments, the shell of the particle typically dissolves prior to the release of the drug. The thickness of the shell can control the time over which its dissolution occurs, and so the delay time for such composite core-shell particles can be varied by varying the shell thickness.

When present, the shell may have any suitable thickness. In some embodiments, the shell of the composite core-shell particles may have a thickness on the order of microns or tens of microns. In certain embodiments, the shells may have a thickness of greater than or equal to 0 microns, greater than or equal to 5 microns, greater than or equal to 10 microns, greater than or equal to 20 microns, greater than or equal to 30 microns, greater than or equal to 40 microns, or greater than or equal to 50 microns. In some embodiments, the shells may have a thickness of less than or equal to 60 microns, less than or equal to 50 microns, less than or equal to 40 microns, less than or equal to 30 microns, less than or equal to 20 microns, less than or equal to 10 microns, or less than or equal to 5 microns. Combinations of the above-referenced ranges are also possible (e.g., greater than or equal to 0 microns and less than or equal to 60 microns). Other ranges are also possible. The thickness of the shell may be measured by scanning electron microscopy.

Those of ordinary skill in the art would be knowledge of techniques to form a polymer shell (e.g., a shell comprising one or more second polymer(s)) on a polymer matrix (e.g., a matrix comprising one or more first polymer(s), a polymer particle, a polymer particle comprising one or more first polymer(s)). For example, a shell may be formed around the polymer matrix using a coaxial double needle geometry comprising an interior needle portion surrounded by an exterior needle portion. The polymer matrix or precursor may be in the interior needle portion and the shell material may be in the exterior portion, such that a droplet released from the needle comprises the polymer matrix or precursor at least partially surrounded (e.g., completely surrounded) by the shell material. In some such embodiments, the polymer matrix precursor and/or shell material may be cross-linked by exposing the droplet to a cross-linking agent (e.g., divalent ion).

As described above, certain methods for forming polymer shells (e.g., shells comprising one or more second polymer(s)) around polymer matrices may be especially advantageous. One such method comprises incorporating one or more shell-forming polymer(s) (e.g., one or more second polymers) into a composition and then transforming that composition into a core-shell composite particle (e.g., by performing one or more method steps described herein). The shell-forming polymer(s) may be polymer(s) that migrate to an external surface of the core-shell composite particle (e.g., from an interior of the core-shell composite particle) as the core-shell composite particle forms and/or forms a shell around the core-shell composite particle as the core-shell composite particle forms. For example, one or more shell-forming polymer(s) may be incorporated into an emulsion, and the emulsion may then be subject to further processing steps that result in the formation of a core-shell composite particle comprising a shell that includes the shell-forming polymer(s).

In certain methods described herein, an agent (e.g., a hydrophobic drug) may be crystallized within a cross-linked emulsion (e.g., a cross-linked emulsion comprising one or more first polymer(s)), and the cross-linked emulsion may comprise at least one polymer which is uncross-linked (e.g., one or more second polymer(s), one or more shell-forming polymer(s)). In some, but not necessarily all, embodiments the uncross-linked polymer may be PVA. The uncross-linked polymer may be preferentially located at the interface between a first solvent and a second solvent (e.g., between a non-aqueous carrier and an aqueous carrier) after the cross-linked emulsion has formed. During the drying process, at least some of the uncross-linked polymer may move from the interface between the non-aqueous carrier and the aqueous carrier to the surface of particles comprising a dispersion of the agent in a cross-linked matrix.

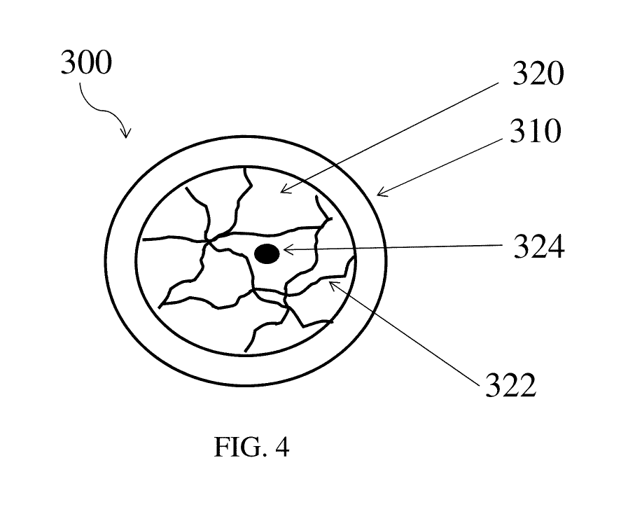

A non-limiting example of a composite core-shell particle in accordance with certain embodiments of the invention is shown in FIG. 4. Composite core-shell particle 300 comprising shell 310 and core 320. Shell 310 may comprise poly(vinyl alcohol) (not shown). Core 320 comprises cross-linked polymer 322 and crystalline hydrophobic drug 324.

It should also be understood that composite core-shell particles, such as those shown in FIG. 4, may comprise any of the polymer matrices (e.g., first polymers), second polymers (e.g., shell-forming polymers), and agents (e.g., hydrophobic drugs) as described above in relation to the emulsions, nanoemulsions, and cross-linked emulsions associated with the inventive methods described herein. In some embodiments, the composite core-shell particles may also comprise water.

As used herein, the core refers to the central portion of the particle and any material topologically connected to the central portion which has a substantially similar chemical composition to the central portion of the particle; the shell refers to the surface of the particle and any material topologically connected to the surface which has a substantially similar chemical composition to the surface of the particle.

In general, any suitable polymer molecules (e.g., first polymer(s)) may be used to form the polymer matrices. In some embodiments, the polymer molecules may be selected based on the intended use of the agent. For instance, in some embodiments, the polymer molecule may be selected based on its compatibility with pharmaceutical applications and other consumer products (e.g., cosmetics, food).

The polymer molecules (e.g., first polymer(s)) are generally extended molecular structures comprising backbones which optionally contain pendant side groups, wherein the term backbone is given its ordinary meaning as used in the art, e.g., a linear chain of atoms within the polymer molecule by which other chains may be regarded as being pendant. Typically, but not always, the backbone is the longest chain of atoms within the polymer. A polymer may be a co-polymer, for example, a block, alternating, or random co-polymer. A polymer may also comprise a mixture of polymers. In some embodiments, the polymer may be acyclic or cyclic. A polymer may be cross-linked, for example through covalent bonds, ionic bonds, hydrophobic bonds, and/or metal binding. Polymer molecules may be obtained from natural sources or be created synthetically.

An exemplary, non-limiting list of polymer molecules that are potentially suitable for use in the invention (e.g., as first polymer(s)) includes polysaccharides (e.g., alginate); polynucleotides; polypeptides; peptide nucleic acids; polyurethane; polyamides; polycarbonates; polyanhydrides; polydioxanone; polyacetylenes and polydiacetylenes; polyphosphazenes; polysiloxanes; polyolefins; polyamines; polyesters; polyethers; poly(ether ketones); poly(alkaline oxides); poly(ethylene terephthalate); poly(methyl methacrylate); polystyrene; poly(lactic acid)/polylactide; poly(glycolic acid); poly(lactic-co-glycolic acid); poly(caprolactone); polysaccharides such as starch; poly(orthoesters); poly(anhydrides); poly(ether esters) such as polydioxanone; poly(carbonates); poly(amino carbonates); and poly(hydroxyalkanoates) such as poly(3-hydroxybutyrate) and poly(3-hydroxybutyrate-co-3-hydroxyvalerate) and derivatives and block, random, radial, linear, or teleblock copolymers, cross-linkable materials such as proteinaceous materials and/or blends of the above. Also suitable are polymer molecules formed from monomeric alkylacrylates, alkylmethacrylates, alpha-methylstyrene, vinyl chloride and other halogen-containing monomers, maleic anhydride, acrylic acid, acrylonitrile, and the like. Monomers can be used alone, or mixtures of different monomers can be used to form homopolymers and copolymers. Other potentially suitable polymer molecules are described in the Polymer Handbook, Fourth Ed. Brandrup, J. Immergut, E. H., Grulke, E. A., Eds., Wiley-Interscience: 2003, which is incorporated herein by reference in its entirety.

The polymer molecules (e.g., first polymer(s)) may have any suitable molecular weight. For example, in some embodiments, the polymer molecules may have an average molecular weight greater than 1000 Da, in certain embodiments greater than 5000 Da, in certain embodiments greater than 10000 Da, in certain embodiments greater than 20000 Da, in certain embodiments greater than 50000 Da, in certain embodiments greater than 100000 Da, in certain embodiments greater than 500000 Da, or in certain embodiments greater than 1000000 Da. In some embodiments, the polymer molecules may have at least 5 subunits, in certain embodiments at least 10 subunits, in certain embodiments at least 20 subunits, in certain embodiments at least 30 subunits, in certain embodiments at least 50 subunits, in certain embodiments at least 100 subunits, in certain embodiments at least 500 subunits, in certain embodiments at least 1000 subunits, or in certain embodiments at least 5000 subunits.

In some embodiments, polymer molecules (e.g., first polymer(s)) may be biodegradable. In other embodiments, a polymer may be non-degradable. In embodiments where the polymer matrices are to be comprised in a composition for administration to a subject, the polymer molecules may be non-toxic, bioabsorbable, and/or unmodified or modified a naturally occurring polymer molecule (e.g., from a plant, from an animal).

In some cases, the polymer molecule (e.g., first polymer) may form a gel or hydrogel (e.g., at the conclusion of a cross-linking process). As used herein, a gel refers to a species which comprises both a polymer and a solvent and which exhibits a yield strength. Hydrogels are gels for which the solvent is at least 85 wt % water. A hydrogel may comprise a network of polymer chains in an aqueous dispersion medium. In some embodiments, a hydrogel may comprise a plurality of cross-linked polymer molecules. In some cases, a hydrogel polymer matrix is formed by cross-linking the polymer molecules. In some embodiments, cross-linking may occur by exposing a cross-linkable polymer (e.g., a cross-linkable agent) to a cross-linking agent. The cross-linking agent may interact with the polymer such that cross-links are formed. This may include forming one or more bonds between at least a portion of the added cross-linking agent and one or more polymer chains. In some embodiments, ionic cross-links may be formed between cross-linking agents with either a positive charge or a negative charge and portions of the polymer with the opposite charge.

In certain embodiments, the cross-linking agent has a positive charge. In some embodiments, the positively charged cross-linking agent comprises ions with a positive charge of greater than +1, such as +2, +3, or +4. The positively charged ions may comprise alkaline earth metal cations, such as Ca⁺².

In some embodiments, the first polymer comprises functional groups with a negative charge. In certain embodiments, the functional groups with a negative charge may comprise deprotonated acids, such as deprotonated carboxylic acids.

Non-limiting examples of polymer molecules (e.g., first polymer(s)) capable of forming gels and/or hydrogels (e.g., by undergoing cross-linking) include: polymers of biological origin such as polysaccharides (e.g., alginic acid, in the acid form and/or as an alginate salt such as sodium alginate), silicon-containing polymers, polyacrylamides, cross-linked polymers (e.g., polyethylene oxide, polyAMPS and polyvinylpyrrolidone), polyvinyl alcohol, acrylate polymers (e.g., sodium polyacrylate), and copolymers with an abundance of hydrophilic groups. In some cases, the polymer molecules may form an organogel, such that the resulting polymer matrices may be swollen by addition of an organic solvent. In some cases, the polymer matrices are a plurality of porous hydrogel particles. In general, the polymer matrix may be formed by cross-linking the polymer molecules. In general, any suitable cross-linking method may be used. For instance, charged polysaccharides (e.g., alginate) may be ionically cross-linked to form a polymer matrix. Those of ordinary skill in the art would be knowledge of suitable cross-linking methods.

In some embodiments, the polymer molecules (e.g., first polymer molecule(s)) may form a gel. A gel may comprise polymer molecules that may be cross-linked to form a network, and the network may be able to trap and contain fluids. Depending on the level of cross-linking, various properties of a particular gel can be tailored. For example, a highly cross-linked gel may generally be structurally strong and may resist releasing fluid under pressure. Those of ordinary skill in the art would be able to identify methods for modulating the degree of cross-linking in such gels.

In some embodiments, the polymer molecules (e.g., first polymer molecule(s)) may comprise functional groups capable of interacting with another polymer molecule and/or a cross-linking agent. In general, the polymer molecules may have any suitable functional groups.

In general, a wide variety of agents may be crystallized using the methods, described herein. In some embodiments, the agent is a molecular species used in consumer products, such as pharmaceuticals, cosmetics, and/or food products. In some embodiments, the agent is a small molecule (e.g., organic), inorganic salt, a macromolecule, biomolecules (e.g., protein, enzyme), and/or combinations thereof.

The methods, compositions, and/or systems of the present invention may find application relating to pharmaceutical compositions and/or methods, when the agent is a pharmaceutically active agent (e.g., a hydrophobic drug). The composition may be isolated and used in a variety of application, e.g., use in a pharmaceutical composition for administration to a subject. In addition, a pharmaceutically active agent primarily encapsulated in a particulate polymer carrier, as described herein, may be used directly in a pharmaceutical composition, reducing or eliminating typical processing steps. For example, the particulate carriers may be bound to form a tablet.

The resulting pharmaceutical composition may be provided to a subject. In some cases, prior to administration to the subject, the pharmaceutical composition may be formed into a pharmaceutical product suitable for administration. For example, the particles may be contained in a capsule (e.g., including gel capsules), as a tablet, in a solution (e.g., for injection), etc.

In some embodiments, methods are provided for administering the particulate polymer carriers comprising a pharmaceutically active agent to a subject. In some cases, the method comprises providing crystals of a pharmaceutically active agent primarily encapsulated in the particulate polymer carriers, due to crystallization of the agent in the presence of the carrier; and administering the plurality of particulate polymer carriers to the subject (e.g., a human).

The term “small molecule” is art-recognized and refers to a composition which has a molecular weight of less than about 2000 g/mole, or less than about 1000 g/mole, and even less than about 500 g/mole. Small molecules may include, for example, nucleic acids, peptides, polypeptides, peptide nucleic acids, peptidomimetics, carbohydrates, lipids or other organic (carbon containing) or inorganic molecules. Another example of a small molecule is fenofibrate (a hydrophobic drug). Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides, or polypeptides. In some cases, the small organic molecule is a pharmaceutically active agent (i.e., a drug, such as a hydrophobic drug). A pharmaceutically active agent may be any bioactive agent. In some embodiments, the pharmaceutically active agent may be selected from “Approved Drug Products with Therapeutic Equivalence and Evaluations,” published by the United States Food and Drug Administration (F.D.A.) (the “Orange Book”). In a particular embodiment, the pharmaceutically active agent is aspirin or acetaminophen.

The compositions and/or crystals described herein may be used in “pharmaceutical compositions” or “pharmaceutically acceptable” compositions, which comprise a therapeutically effective amount of one or more of the polymers or particles described herein, formulated together with one or more pharmaceutically acceptable carriers, additives, and/or diluents. The pharmaceutical compositions described herein may be useful for diagnosing, preventing, treating or managing a disease or bodily condition including conditions characterized by oxidative stress or otherwise benefitting from administration of an antioxidant. Non-limiting examples of diseases or conditions characterized by oxidative stress or otherwise benefitting from administration of an antioxidant include cancer, cardiovascular disease, diabetes, arthritis, wound healing, chronic inflammation, and neurodegenerative diseases such as Alzheimer Disease.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid, gel or solid filler, diluent, excipient, or solvent encapsulating material, involved in carrying or transporting the subject compound, e.g., from a device or from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, a “subject” or a “patient” refers to any mammal (e.g., a human), for example, a mammal that may be susceptible to a disease or bodily condition. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition.

Those of ordinary skill in the art can select suitable substantially immiscible fluids, using contact angle measurements or the like, to carry out the techniques of the invention.

A “droplet,” as used herein, is an isolated portion of a first fluid that is completely surrounded by a second fluid. In some cases, the first fluid and the second fluid are substantially immiscible. It is to be noted that a droplet is not necessarily spherical, but may assume other shapes as well, for example, depending on the external environment. The diameter of a droplet, in a non-spherical droplet, is the diameter of a perfect mathematical sphere having the same volume as the non-spherical droplet. The droplets may be created using any suitable technique, as previously discussed.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

The following examples are intended to illustrate certain embodiments of the present invention, but do not exemplify the full scope of the invention.

### EXAMPLES

Nucleation of crystalline materials is omnipresent in nature and industrial practice, specifically, in the chemical and pharmaceutical industry. A promising direction for controlling crystallization is to target nucleation, a critical step in the crystallization process, by designing heteronucleant materials capable of influencing crystallization through selective interactions. However, in industrial practice particularly in pharmaceutical industry, moieties to be crystallized are diverse in chemical structure and accordingly in physical properties such as solubility. The diversity of the moieties along with the demand encountered in industrial practice for biocompatible crystallization promoters introduces additional constraints on the design of biocompatible materials capable of influencing nucleation behavior, crystal formation, crystal size, and morphology. The heteronucleant material designed for industrial practice should be biocompatible, capable of controlling crystallization, capable of carrying industrially relevant amounts of crystalline material (e.g. pharmaceutical in crystalline form), and applicable to hydrophobic and hydrophilic moieties. These examples describe a composite biocompatible hydrogel capable of controlling nucleation from solution, due to rational design of the microstructure and chemical makeup of hydrogel particles, and capable of carrying industrially relevant amounts of water soluble and insoluble pharmaceuticals in chemically distinct environments within composite hydrogel.

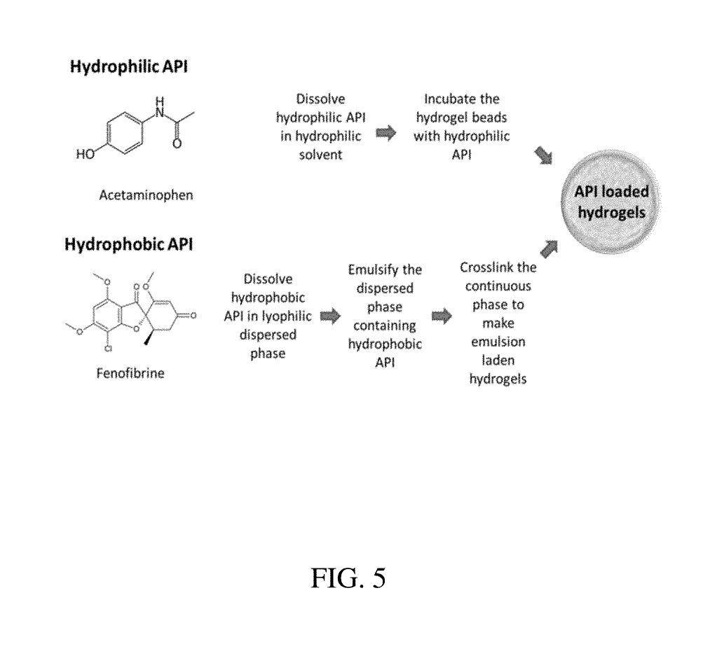

In these examples, methods for designing nanostructure and chemical makeup of biocompatible alginate (ALG) hydrogel particles capable of (i) controlling nucleation from solution and (ii) carrying crystalline active pharmaceutical ingredients (API) of diverse chemical nature (iii) controlling size of the crystals in the final formulation are described. Additionally, the control of nucleation kinetics of the model hydrophilic active pharmaceutical ingredient (Acetaminophen, ACM) from solution and encapsulation of the active pharmaceutical ingredient into the hydrogel through equilibrium partitioning as a function of mesh size is demonstrated. A hydrophobic model active pharmaceutical ingredient (Fenofibrate, FEN) is described that uses, emulsion laden composite hydrogels synthesized for both encapsulation and crystallization. The composite hydrogels were capable of encapsulating and crystallizing the hydrophobic active pharmaceutical ingredient inside emulsion droplets and carrying industrially relevant amounts of active pharmaceutical ingredient. Furthermore, the size of crystals was controlled by adjusting the droplet size and concentration of active pharmaceutical ingredient. FIG. 5 is a schematic illustration of the methods used to form hydrogels loaded with hydrophobic active pharmaceutical ingredients and hydrogels loaded with hydrophilic active pharmaceutical ingredients.

### Example 1

This example describes the synthesis formation of biocompatible hydrogels with controlled microstructure. In an effort to create a pharmaceutically acceptable nucleation step active hydrogel capable of carrying active pharmaceutical ingredients of distinct polarities, a biocompatible polysaccharide used in food, and pharmaceutical industry isolated from brown algae, alginate (ALG) was investigation. Alginate is a linear copolymer, consisted of b-D-mannuronic acid (M) and its C-5 epimer, a-L-guluronic acid (G), arranged in a blockwise pattern. Alginate gel formation can be induced by lowering pH or by adding various divalent cations, in particular Ca⁺², which cross-links a pair of G blocks within the alginate chains. Despite its utility for carrying hydrophilic drugs, alginate has a hydrophilic nature and accordingly hydrophobic drugs cannot be solubilized or loaded into hydrogel, such alginate, in hydrophilic solvents. To overcome this limitation, composite hydrogels were designed by introducing hydrophobic regions inside hydrogel network by encapsulated nanoemulsion droplets in the hydrogel network. The nanoemulsions provide isolated microenviroments that are chemically different than their surrounding network.

### Example 2

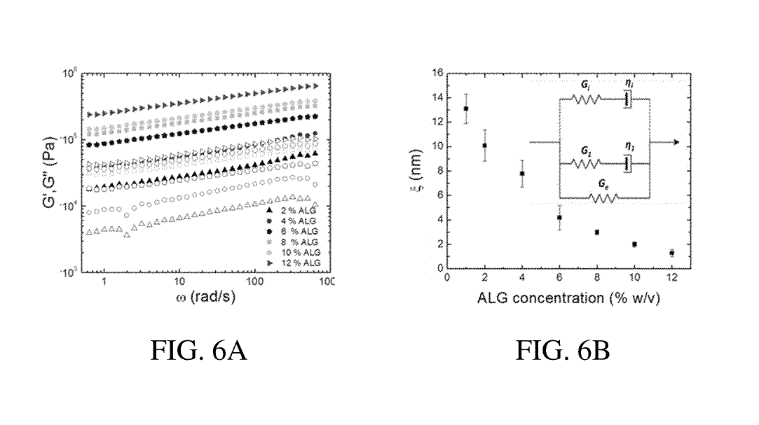

This example describes the characterization of alginate hydrogel formed as described in Example 1. Alginate hydrogels were characterized by evaporation to measure relative solvent content and rheological measurements to estimate the mesh size. For the evaporation measurements, spherical alginate shaped beads (FIG. 6A) were used whereas for rheological measurements disk shaped hydrogels were prepared due to the requirements of the rheometer. Both hydrogel geometries were Ca²⁺ saturated to completely cross-link alginate hydrogels. The evaporation measurements provided information on the weight ratio of solvent to hydrogel bead which was then converted to the volume ratio of hydrogel as the volume of the bead was known a priori. To determine the weight ratio, the hydrogels beads (approximately 20 beads) were first pat dried and weighed and then a microscope image was taken to estimate the size of the hydrogel beads (FIG. 6A). The beads were then placed in a vacuum oven set to 120° C. over night to evaporate the solvent. The weight of the dried hydrogel was measured after evaporation. The difference between the weight of the wet hydrogel and the dry hydrogel was recorded as the weight of evaporated solvent. The weight of the evaporated solvent was converted to volume of evaporated solvent as the density of the solvent was known. The weight and volume ratio of the solvent to the alginate is given in FIG. 6B. All the measurements are performed in triplicates. FIG. 6A shows rheological measurements for alginate hydrogels with different alginate concentrations. FIG. 6B shows the average mesh size based on alginate concentration and a mechanical illustration of Maxwell model for calculating mesh size (inset).

The characterization of mesh size for alginate hydrogels with different alginate concentrations was performed by oscillatory rheology. Disk shaped alginate gels with different alginate concentrations, namely, 2, 4, 6, 8, 10 and 12% (w/v), were prepared in calcium-saturated conditions and their rheological response was recorded (FIG. 6A). In all alginate concentrations considered, the storage (G′) modulus was monotonous and significantly higher than the loss modulus (G″), indicating strong gels behavior. Frequency sweep measurements at a fixed stain (0.05%) were modeled in terms of the generalized Maxwell model. The model utilized was composed of a sequence of elements in parallel (spring and dashpot) to which an additional spring has been added (FIG. 6B). The use of the generalized Maxwell model to describe the alginate system allowed the shear modulus, G and mesh size (ξ) to be determined.

### Example 3

This example describes a method of controlling the nucleation kinetics of hydrophilic active pharmaceutical ingredient by adjusting hydrogel mesh size. No study has been reported on controlling kinetics of nucleation from solution with polymers of tunable nanostructure. Moreover, the effects of pore sizes on the rate of nucleation from solution have not been experimentally studied. In addition, the effect of pore chemistry on nucleation has been largely neglected. Overall, mechanistic understanding of nucleation from solution in nanoconfinement required the design polymers with the proper nanostructure and chemistry to control crystallization is inadequate.

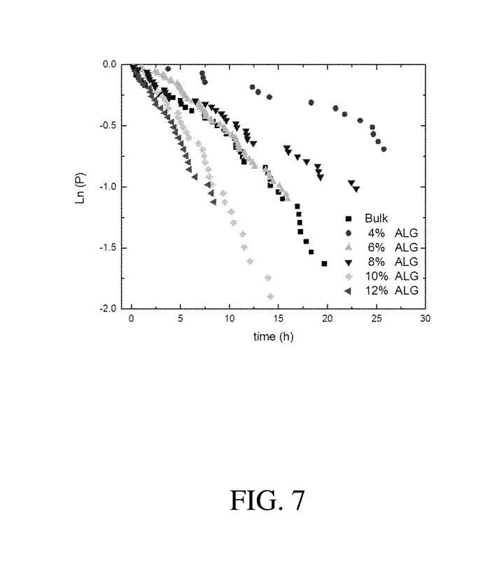

To enable rational design of hydrogels for controlling crystallization of hydrophilic active pharmaceutical ingredient the effect of mesh size on nucleation kinetics was systematically investigated by changing the mesh size via polymer concentration as seen in FIG. 7. In the crystallization solvent, the mesh sizes of these hydrogels ranged from 1 nm to 12 nm. It was found that by changing the mesh size of hydrogel a nucleation event could be enhanced with respect to bulk nucleation. Thus, tuning the microstructure of hydrogels can be used to enhance nucleation from solution.

FIG. 7 shows a graph of time as a function of the natural logarithm of the nucleation induction probability for crystallizations in the presence of alginate particles at different percent composition (4%, 6%, 8%, 10%, and 12%) and the absence of alginate particles versus time.

Nucleation kinetics of model compounds was investigated by measuring the nucleation induction time probability distribution. As used in this example, nucleation induction time is defined as the time elapsed prior to the formation of a detectable amount of the new crystalline phase. Without being bound by theory, it is believed that nucleation induction time is a useful indicator of the surface nucleation activity because nucleation induction time can be dramatically shortened when the presence of an interface lowers the free energy barrier of nucleation. A large number of experiments were performed to obtain the probability distribution of nucleation induction time. To obtain the average induction time T, statistical analysis on the induction time data was conducted based on the understanding that nucleation follows a Poisson distribution. According to Poisson statistics, the probability for a nucleation event to occur beyond time t is P=exp(−t/τ), which implies that the fraction of vials without nucleation at time t exponentially decays as a function of time, where the scaling factor for time is the average induction time. The induction times are given in Table 1.

TABLE 1
Summary of average induction time (τ) measurements for
alginate particles of different concentrations used as
heteronucleants during crystallization of ACM from a
supersaturated ethanol solution at 10° C.

| sample | τ (hrs.) | error (hrs.) | linearity |
|---|---|---|---|
| control | 15.3 | 0.31 | 0.99 |
| 4% alginate | 24.7 | 1.4 | 0.92 |
| 6% alginate | 12.8 | 0.23 | 0.97 |
| 8% alginate | 16.5 | 0.50 | 0.95 |
| 10% alginate | 9.5 | 0.18 | 0.98 |
| 12% alginate | 7.7 | 0.10 | 0.99 |

### Example 4

Example 4 describes a method of controlling the loading of hydrophilic active pharmaceutical ingredient via mesh size of hydrogel.

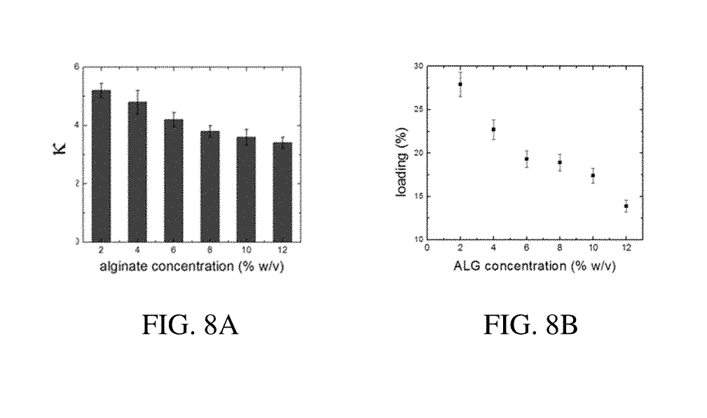

It has been demonstrated that hydrophilic active pharmaceutical ingredient partitioning into alginate hydrogel can be controlled by polymer concentration (% alginate by weight prior to cross-linking) i.e., mesh size of hydrogel (FIG. 8A and FIG. 8B show the ACM loading in hydrated hydrogels calculated by equilibrium partitioning for various alginate concentrations and the percent loading of active pharmaceutical ingredient in alginate hydrogel in weight percent). The hydrogels immersed in a polar solvent in equilibrium with solid active pharmaceutical ingredient could load up to 27 wt. % of hydrophilic active pharmaceutical ingredient through equilibrium partitioning. To determine the amount of drug loaded a known amount of the particles was transferred to a known amount of solvent (water) and stirred at a constant temperature. The dissolution or release of the drug was monitored over time using UV-vis spectrometry and the concentration of the drug at equilibrium was determined using its absorbance. It was observed that the amount of drug loaded at lower alginate concentration was more than twice the amount loaded at higher alginate concentrations (FIG. 8). Without being bound by theory, it is believed that this effect might be due to the larger pore size and size distribution of the lower alginate concentrations and also the solute partition coefficient which decreases with increasing alginate concentration.

### Example 5

This example describes the formulation of composite hydrogels for crystallizing hydrophobic active pharmaceutical ingredient.

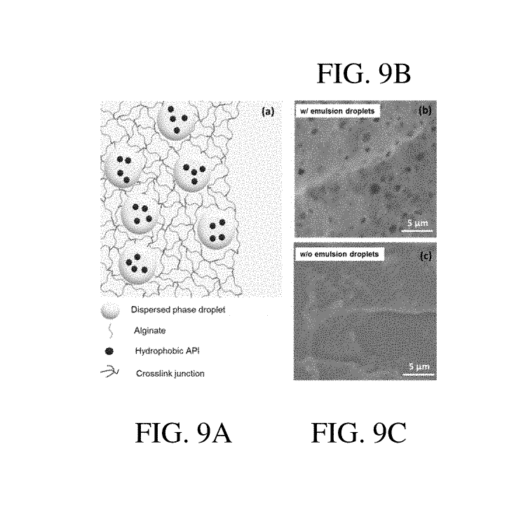

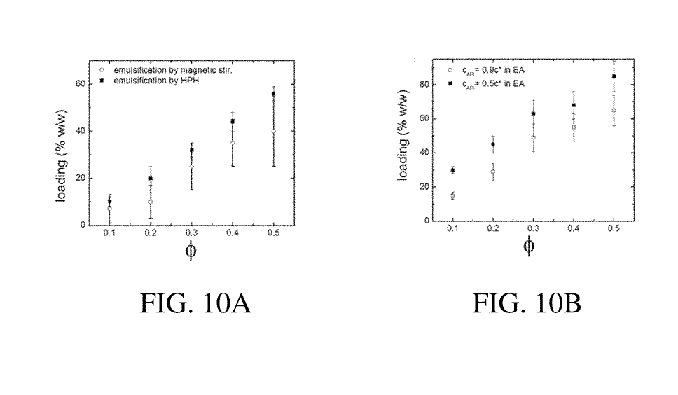

Composite hydrogels containing emulsion droplets were embedded in hydrogel matrix, effectively creating hydrophobic regions in an otherwise hydrophilic alginate network. The dispersed phase was emulsified in nano and micron size droplets using heptane and ethyl acetate as a solvents in order to load the drugs inside the alginate particles (FIG. 9A shows an illustration describing emulsion laden composite hydrogels loaded with hydrophobic active pharmaceutical ingredient, fenofibrate (FEN). Environmental scanning electron microscope images confirmed the presence of the emulsified droplets inside the alginate particle (FIG. 9B-C show cross-section of an environmental scanning electron microscopy (eSEM) images of alginate hydrogels (B) with emulsion droplets and (C) without emulsion droplets serving as control for panel (B). The continuous phase contained 2% alginate and the volume fraction of the dispersed phase was 30% (v/v) FEN in heptane). The hydrophobic compound utilized as a model active pharmaceutical ingredient for the hydrophobic drugs was fenofibrate (FEN). The emulsified solution was added to alginate solutions having various concentrations. The emulsified solution containing the fenofibrate and alginate solution was cross-linked in situ in a calcium chlorine solution. The emulsion laden composite hydrogels were formed with different emulsion volume fractions ranging between 10% to 50% (FIG. 10). The emulsion laden composite hydrogels had the ability to crystallize FEN inside droplets and also demonstrated tunable loading up to 85% by weight active pharmaceutical ingredient in dried hydrogel.

### Example 6

This example describes the loading characteristics of composite hydrogel with respect to hydrophobic active pharmaceutical ingredients.

The utility of loading industrially relevant amounts of hydrophobic drug and the ability to control the amount of drug loaded by varying volume fraction of dispersed phase, dispersed phase chemical makeup, and concentration of active pharmaceutical ingredient in dispersed phase was demonstrated. First the advantage of using high pressure homogenization (HPH) for emulsification step (FIG. 10 was demonstrated). In FIG. 10A, it is shown that with HPH, a smaller variation in loading was achieved compared to emulsification with magnetic stirring. This was attributed to smaller variations in droplet size obtained in HPH than magnetic stirring, therefore it was concluded that HPH was a better suited for pharmaceutical production where dosage consistency is of utmost importance.

Secondly, the ability to control amount of hydrophobic drug loaded in hydrogel on dry basis by changing volume fraction and concentration of active pharmaceutical ingredient in dispersed phase (Ethyl Acetate (EA)) was demonstrated. For each loading measurement of a given volume fraction (φ), two batches of emulsion laden hydrogels were prepared with the same method of emulsification followed by cross-linking (i.e., a reference batch without active pharmaceutical ingredient and a test batch with active pharmaceutical ingredient). Both batches contained approximately 200 mg of alginate beads and were pat-dried and weighted. Both batches were placed in vacuum oven and dried over two days at 140° C. which is above the boiling point of dispersed phase (boiling point of heptane 98° C., ethyl acetate 77.1° C. at 1 bar). Initially heptane was used as the continuous phase; later in an effort to maximize the loading ethyl acetate was used as the continuous phase. The solubility of FEN in ethyl acetate was considerably larger than in heptane (9 mg/mL and 600 mg/mL). Loading was defined as the difference in weight between the hydrogels containing the active pharmaceutical ingredient carrying emulsions and reference batch the without the active pharmaceutical ingredient divided by weight of active pharmaceutical ingredient carrying emulsion laden hydrogel on dry solute basis

$$as \text{:} Loading \% = \frac{{W}_{API carrying emulsion laden hydrogel} - {W}_{reference emulsion laden hydrogel}}{{W}_{API carrying emulsion laden hydrogel}} * 100.$$

To estimate the variation in loading and significance of variation in ten replicates were performed.

FIG. 10A is a graph showing the loading of FEN in heptane emulsion laden hydrogels measured by evaporation method. FIG. 10B is a graph showing the loading of FEN in ethyl acetate with two different FEN concentrations for varying emulsion volume fractions measured by evaporation method.

### Example 7

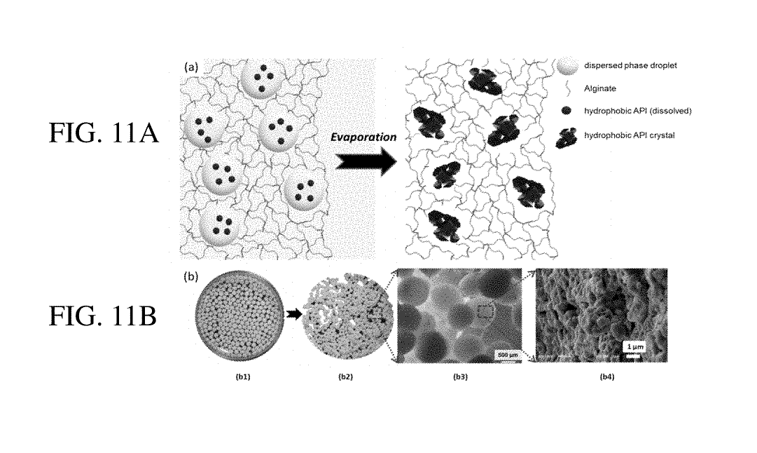

This example describes the control of crystal size using composite hydrogels. The distribution in crystal size of pharmaceuticals in a formulation is critical to the pharmaceuticals dissolution rate and accordingly pharmokinetic performance in body. Smaller crystals provide a larger surface area for a given active pharmaceutical ingredient mass. The larger area promotes quicker dissolution allowing active pharmaceutical ingredient to dissolve more quickly in vivo. For hydrophobic drugs with low aqueous solubility, there are limited methods for achieving a reduced crystal size. The state of the art methods such as mechanical milling or spray freezing into liquid are harsh treatments that can induce formation of metastable polymorphs. In this example, the development of composite hydrogel for controlling crystal size is described and the demonstrated control of the dissolution rate for an hydrophobic active pharmaceutical ingredient is demonstrated. The composite hydrogels consisted of trapped emulsion droplets carrying active pharmaceutical ingredient in a biocompatible polymer (alginate) matrix. By controlled evaporation of continuous phase and dispersed phase, crystallization of active pharmaceutical ingredient with in hydrogel matrix was induced (FIG. 11). Control of emulsion droplet size and active pharmaceutical ingredient concentration within the emulsion droplet allowed crystals as small as 300 nm and as large as 0.5 mm to be produced.

FIG. 11 shows a pictorial demonstration of producing embedded crystals in dried hydrogel matrix through controlled evaporation of composite hydrogel where the crystal size was controlled by droplet volume and concentration of active pharmaceutical ingredient. First hydrophobic active pharmaceutical ingredient (FEN) was dissolved in an organic phase (FDA Class III solvent Anisole). Then the organic phase carrying the hydrophobic active pharmaceutical ingredient was dispersed in an aqueous continuous phase with a suitable surfactant (Pluronic® F-68). Due to preferential partitioning of the hydrophobic active pharmaceutical ingredient to the dispersed organic phase, the active pharmaceutical ingredient was predominantly in the organic phase; only a minute fraction of the active pharmaceutical ingredient partitioned into the continuous aqueous phase. The Na-alginate dissolved in continuous phase was then ionically cross-linked with Ca²⁺ ions in order to trap dispersed phase droplets containing the active pharmaceutical ingredient in the organic phase in the hydrogel matrix. The trapped dispersed phase inside the hydrogels matrix formed composite hydrogels depicted in FIG. 11A.

Through controlled evaporation at 60° C., evaporation of both the dispersed phase and continuous phase was induced. Once all liquids were evaporated the crystals were entrapped in dried hydrogel matrix. The crystals were larger than the mesh size of the polymer matrix, but could not grow above the size of emulsion due to confinement. FIG. 11B shows the composite hydrogels in hydrated form (b1), after drying (b2) optical microscope image of dried composite beads (b3), and a high magnification SEM image of dried composite hydrogel sliced cross-section (b4).

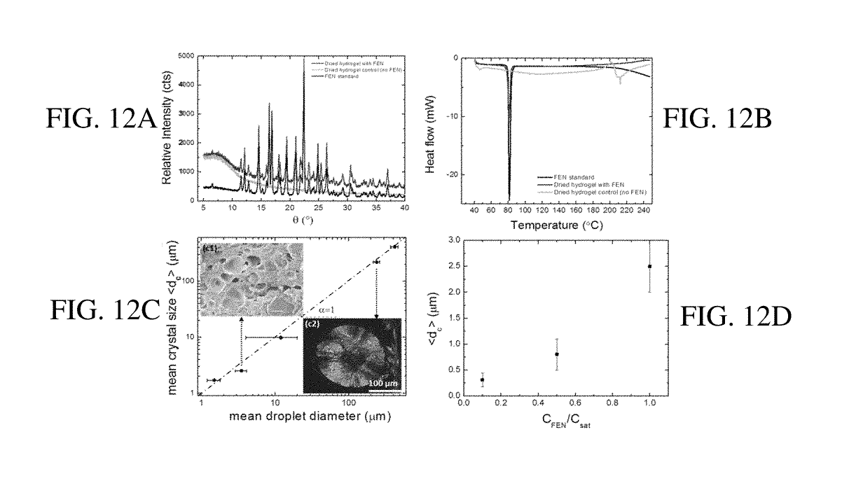

To investigate if the active pharmaceutical ingredient was crystalline inside the dried composite hydrogel, powder X-ray diffraction scattering (PXRD) and differential scanning calorimetry (DSC) was used. FIG. 12A shows PXRD pattern from the FEN standard, dried composite hydrogel carrying FEN and control dried composite hydrogel without FEN. The XRD patterns of standard and dried composite hydrogel carrying FEN indicated the crystalline structure of FEN inside dried hydrogels. DSC also showed FEN standard, dried composite hydrogel carrying FEN and control dried composite hydrogel without FEN. The melting point of FEN standard and dried composite hydrogel carrying FEN coincided at 81° C. (FIG. 12B). These findings together with PXRD results proved that FEN was crystalline inside dried composite hydrogels.

The crystal size was controlled by the droplet size and the concentration of the active pharmaceutical ingredient in dispersed phase. When the dispersed phase was saturated with the active pharmaceutical ingredient (i.e., concentration of FEN in dispersed phase is equal to saturation concentration of FEN in the dispersed organic phase (C_{FEN}/Cₛₐₜ=1)), the droplet size of the dispersed phase dictated the size of the crystals. Using three different emulsification techniques, namely high pressure homogenization, bulk emulsification and millifluidics, emulsions ranging between 1.5 micron to 0.5 mm were prepared. As seen in FIG. 12C, the mean droplet size and mean crystal size were equal within the error bounds for three different emulsification techniques. In addition to droplet size, the crystal size was controlled by controlling the concentration of the active pharmaceutical ingredient within the dispersed phase. By decreasing the concentration of C_{FEN} in the dispersed phase, the crystal size was decreased below the droplet size. Hence controlling C_{FEN}/Cₛₐₜ, allowed control over the crystal size and allowed crystals smaller than size dictated by the droplet size to be formed.

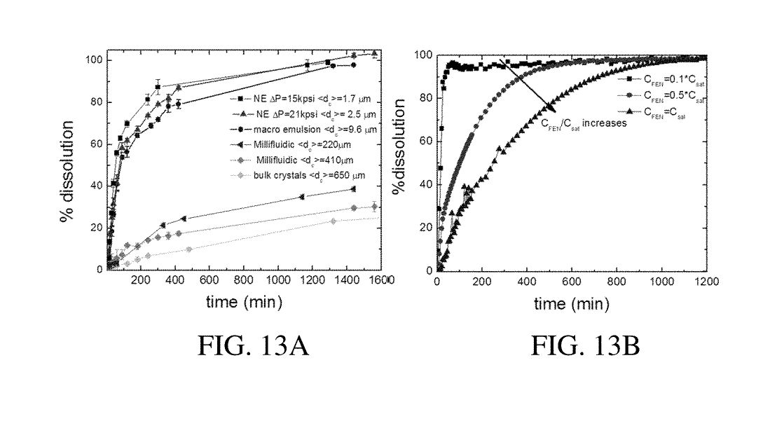

By controlling the crystal size, the dissolution profiles of dried composite hydrogels could also be controlled. Dried composite hydrogels with different crystal sizes exhibited different dissolution profiles. Smaller crystal sizes give larger surface area per unit mass and this led to faster dissolution. FIG. 13A shows dissolution profiles of dried hydrogels with different mean crystal sizes (C_{FEN}/Cₛₐₜ=1). FIG. 13B shows that the dissolution profile could be tuned by controlling concentration of active pharmaceutical ingredient inside emulsion droplets and hence the crystal size. Composite hydrogels that had the same entrapped dispersed phase droplet size but different FEN concentrations resulted in different dissolution rates.

FIG. 13A shows the dissolution profile for different crystal sizes where organic phase was saturated by FEN i.e. C_{FEN}=C_{FEN}^{SAT}. FIG. 13B shows the dissolution profile where the crystal size was controlled by concentration of FEN in emulsion droplets of given sizes.

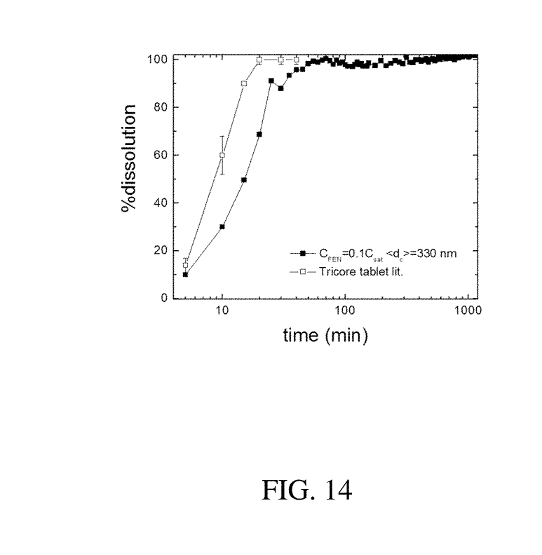

In some embodiments, a fast dissolution time is important. The results above were compared to commercially available formulation TriCor tablets. FIG. 14 shows the semilog plot dissolution profiles of FIG. 13B compared to literature. FIG. 14 shows that the dissolution rates were comparable to commercially available formulations.

The state of the art in industrial practice to control nucleation involves methods such as adjusting supersaturation levels, temperature profiles, crystallization solvent, stirring speed, seeding with existing active pharmaceutical ingredient crystals, etc. However, the nucleation behavior remains largely unpredictable due to the presence of unregulated foreign surfaces that lower nucleation energy barrier. The Examples describe a biocompatible hydrogel excipient particles with morphology designed specifically to directly control the nucleation kinetics and crystal outcome for hydrophobic and hydrophilic active pharmaceutical ingredient. The methodology developed was amenable to continuous manufacturing, particularly composite hydrogels provided compartmentalized units where crystallization could be achieved. These compartmentalized units were accessible as they were embedded in a hydrogel. Hence crystallization could be induced either by temperature shock, evaporation, or chemical interference.

The current state of the art in improving dissolution rates of hydrophobic active pharmaceutical ingredient are based on decreasing crystal size of mm size native active pharmaceutical ingredient crystals with harsh milling or spray drying methods. Due to their harsh nature with considerable energy input, these methods are known to introduce polymorphism i.e. formation of metastable polymorphs. The Examples describe a methodology of using composite hydrogels in mild methods where the active pharmaceutical ingredient crystals were formed inside a hydrogel matrix hence metastable polymorphs were avoided. It was found that the crystal size, which dictates the dissolution profile, could be controlled by controlling droplet size of trapped dispersed phase and concentration of active pharmaceutical ingredient. Relatively high loading of submicron crystals at (up to 20% active pharmaceutical ingredient on dry basis) was also described.

Furthermore, the polymer matrix in which crystals were embedded provided natural protection against mechanical effect that could disturb crystal morphology. Once the crystals were formed they were protected from mechanical effects, because the crystals were surrounded by the hydrogel matrices. Additional protective layers could be added by using coaxial needles with ease.

The ability of the developed biocompatible hydrogel excipients to carry pharmaceutically relevant amounts of both hydrophobic and hydrophilic active pharmaceutical ingredient was unique. It was demonstrated that the developed alginate hydrogels and composite alginate hydrogels containing nanoemulsion droplets could carry active pharmaceutical ingredient using two separate mechanisms. Alginate hydrogels' ability to absorb and concentrate hydrophilic active pharmaceutical ingredient due to equilibrium partitioning within the interior of the particles was unique. Such partitioning is not readily obtained with any other type of previously used excipient (SAMs, crystalline surfaces, glass, solid polymers, etc.). Conversely, the composite hydrogels containing nanoemulsion droplets were embedded in the hydrogel matrix trapped large amounts of hydrophobic active pharmaceutical ingredient due to lyophilicity of nanoemulsion. Furthermore, aforementioned mechanisms could be utilized to orthogonally load composite hydrogels with industrially relevant (up to 85% by dry weight) amounts of hydrophobic and hydrophilic active pharmaceutical ingredient. Also, nanoemulsions carrying different hydrophobic pharmaceuticals could be loaded inside composite hydrogels. The ability to control crystallization and load large amounts of active pharmaceutical ingredient with diverse solubility, individually or orthogonally, make the polymer matrices, described herein, unique.

### Example 8

This example describes applications in which the methods, compositions, and systems described in Examples 1-7 may be used and are prophetic.

The method described in this invention could be applied to designing biocompatible particles to regulate nucleation kinetics, to load active pharmaceutical ingredient of diverse chemical and physical properties and to crystallize hydrophobic active pharmaceutical ingredient with controlled size (appx. 300 nm to 0.5 mm) for pharmaceutical industry, food industry and other industries that require crystallization and delivery of small organic compounds.

Applications in Pharmaceutical Manufacturing of Hydrophilic Active Pharmaceutical Ingredient

Crystallization is extensively used to purify the active pharmaceutical ingredients in the pharmaceutical manufacturing process. After the crystallization step, the active pharmaceutical ingredient crystals are then granulated and blended with excipients before packaging into the final dosage form. Granulation and blending processes are problematic due to their harsh nature where active pharmaceutical ingredient crystals can be broken or aggregated even transformed into a metastable polymorph. Pharmokinetic performance of pharmaceutical is very sensitive to the shape and size of the drug crystals. Hence manufacturing processes where the crystal size and morphology can be controlled and protected from environmental factors is desired.

The methods, compositions, and systems described in Examples 1-7 allowed heterogeneous crystallization of hydrophilic active pharmaceutical ingredient from solution on the surface of an amorphous excipient, so that the resulting active pharmaceutical ingredient-excipient composite particles could either serve as the dosage form themselves, or were agglomerated and formed directly into the final dosage form, thereby eliminating the subsequent granulation, blending, and compaction steps. Furthermore, design of the excipient surface properties to control the active pharmaceutical ingredient nucleation kinetics and final crystal form allowed for better control over the quality and uniformity of the final drug and dosage form.

Application in Pharmaceutical Manufacturing of Hydrophobic Active Pharmaceutical Ingredient

Since 1980, 43% of all newly developed pharmaceuticals are estimated to be extremely insoluble in water. Hence new methodologies for producing hydrophobic active pharmaceutical ingredient are essential in pharmaceutical industry. The current state of art in production of hydrophobic pharmaceuticals is based on granular particle processing that is by definition a batch process or using organic solvents that needs to be removed prior to final packaging.

The composite hydrogels described in the Examples, namely composite hydrogels containing nanoemulsion droplets, were capable of carrying and crystallizing hydrophobic active pharmaceutical ingredient in aqueous environment. Hence the composite hydrogels either served as the dosage form themselves, or were dried into the final dosage form, eliminating the batch granular particle processing. The nanoemulsion droplets acted as hydrophobic regions favoring hydrophobic active pharmaceutical ingredient within hydrophilic hydrogel. This favorable interaction was responsible for pharmaceutically relevant amount (up to 85% by weight active pharmaceutical ingredient in hydrogel on dry basis) of agent that was loaded.

With controlled mild evaporation, composite hydrogels carrying active pharmaceutical ingredient were transformed into final dosage form with controlled active pharmaceutical ingredient crystal size embedded in dried hydrogel(polymer) matrix. The crystal size were controlled by adjusting droplet size and active pharmaceutical ingredient concentration.

Applications in Hydrophobic Active Pharmaceutical Ingredient Delivery

The current state of the art in pharmaceutical industry for improving dissolution rates of notoriously difficult to dissolve hydrophobic active pharmaceutical ingredient involves reducing the size via nanomilling methods or physical absorption into nanoporous matrix in amorphous state. However, these methods are not ideal because the drugs are prone to phase transformation into metastable polymorphs under mechanical stress or to recrystallize since the amorphous form is metastable. Methods, compositions, and/or systems, described herein, demonstrated direct crystallization of hydrophobic active pharmaceutical ingredient in the drug carrier to a desired crystal size, and accordingly, desired dissolution rate.

### Example 9

This example describes the formation of core-shell composite hydrogel particles from nanoemulsions. Poly(vinyl alcohol) (PVA) is used as a surfactant during nanoemulsion formation, and forms the shell of the resultant particles at the conclusion of the particle formation process.

### Abstract

Although roughly 40% of pharmaceuticals being developed are poorly water-soluble, this class of drugs lacks a formulation strategy capable of producing high loads, fast dissolution kinetics, and low energy input. In this work, a novel bottom-up approach for producing and formulating nanocrystals of poorly water-soluble active pharmaceutical ingredients (APIs) using core-shell composite hydrogel beads is developed. Organic phase nanoemulsion droplets stabilized by polyvinyl alcohol (PVA) and containing a model hydrophobic API (fenofibrate) are embedded in the alginate hydrogel matrix and subsequently act as crystallization reactors. Controlled evaporation of this composite material produces core-shell structured alginate-PVA hydrogels with drug nanocrystals (500-650 nm) embedded within the core. Adjustable loading of API nanocrystals up to 83% by weight is achieved with dissolution (of 80% of the drug) occurring in as little as 30 minutes. A quantitative model is also developed and experimentally validated that shows that the drug release patterns of the fenofibrate nanocrystals can be modulated by controlling the thickness of the PVA shell and drug loading. Thus, these composite materials offer a ‘designer’ drug delivery system. Overall, this approach enables a novel means of simultaneous controlled crystallization and formulation of hydrophobic drugs that circumvents energy intensive top-down processes in traditional manufacturing.

### Results and Discussion

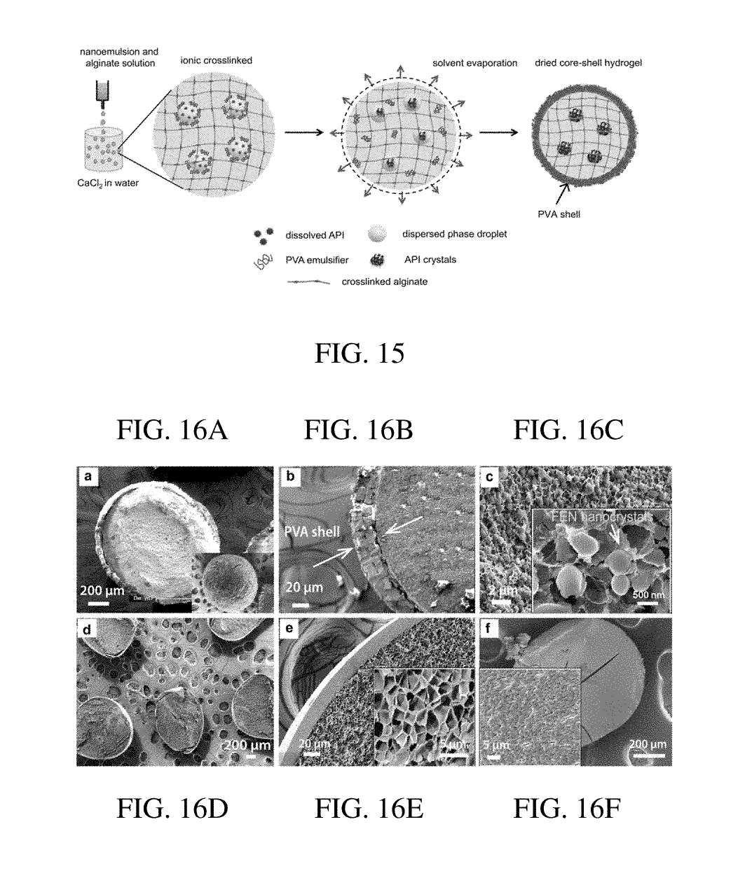

A schematic diagram of the formation of a composite hydrogel with a core-shell microstructure is shown in FIG. 15. Generating the core-shell hydrogel material is a multi-step process that begins with preparing the oil in water nanoemulsions by emulsifying the hydrophobic organic phase (anisole) saturated with FEN in an aqueous solution containing the biocompatible polymer alginate (ALG) and PVA. The PVA is used for stabilizing the nanoemulsion droplets. PVA is an FDA approved GRAS (generally recognized as safe) material and has been widely used as an emulsifier, binder and film coating agent in the food and pharmaceuticals industries. Anisole is chosen as the dispersed phase due to its approval for pharmaceutical manufacturing and the high saturation concentration (C_{satFEN}) of fenofibrate in this solvent (˜400 mg/mL). Nanoemulsions are produced by ultrasonication, which is a robust process for preparing droplets across a range of sizes. ALG beads are produced by dripping the uncross-linked nanoemulsion dispersion into a 6% w/v CaCl₂ bath in drop-wise fashion. The final bead size depends on the composition of the nanoemulsion solutions and the diameter of the needle. Ionic cross-linking of ALG creates a cross-linked polymer network trapping the nanoemulsion droplets containing the API. Crystallization of FEN is induced by controlled evaporation of both the dispersed organic phase and the aqueous phase at 60° C. During this drying process, the PVA molecules migrate from the aqueous-organic interface to the external surface of the beads, which results in the formation of hydrogel beads with a core-shell microstructure. This approach enables the shell thickness to be engineered either by varying the volume fraction of the dispersed phase at constant PVA concentration or by changing the particle size at constant volume fraction. The ability to tune shell thickness offers modulated control of the dissolution behavior of the embedded API, which will be discussed in more detail later.

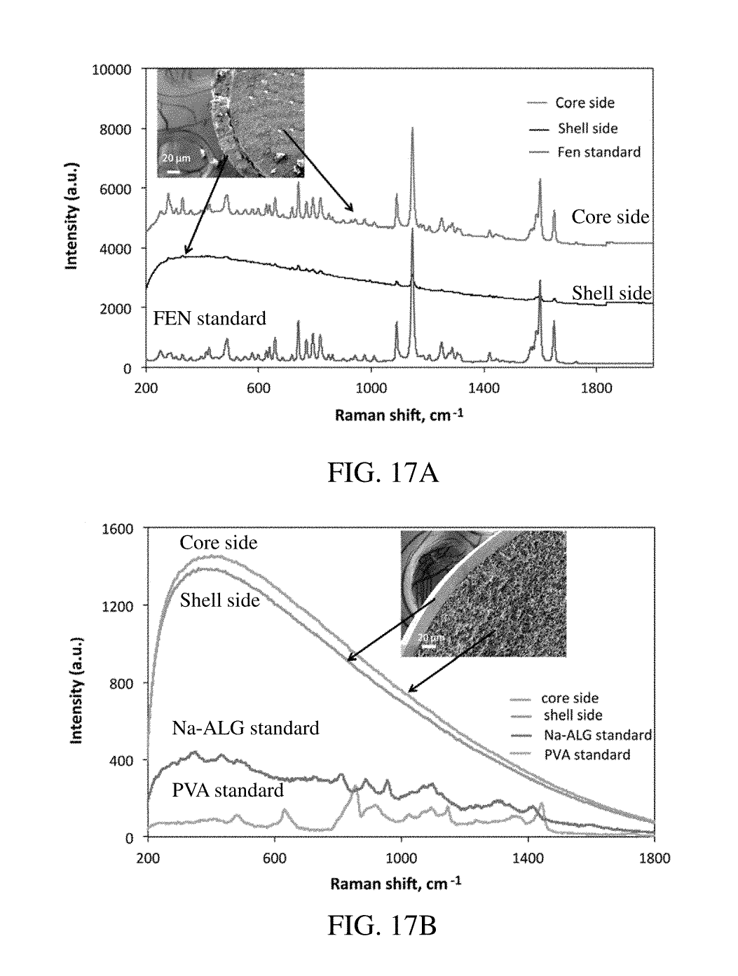

The size, morphology and microstructure of the core-shell composite hydrogels are characterized by high resolution scanning electron microscopy (HR-SEM). FIG. 16 shows SEM images of core-shell composite hydrogels (both with and without API, as a control) representing a typical API nanocrystal formulated microparticle. The composite hydrogel has a core-shell structure obtained at certain experimental conditions (φ=20% dispersed phase with C_{FEN}=C_{satFEN} emulsified in 2% w/v ALG containing 1% w/v PVA in the upper left panel in FIG. 16). The upper right panel in FIG. 16 also shows FEN nanoparticles confined by pores within the ALG core with some vacant cavities. The presence of the FEN nanocrystals embedded inside the core was further confirmed by Raman Spectroscopy (see FIG. 17). The control sample which lacks API (the lower left and center panels of FIG. 16) shows only vacant pores (1 to 2 microns) with no nanocrystals inside, indicating that the porous alginate matrix facilitates confined crystallization. This control experiment also confirms that the API is not necessary for the shell formation. However, the shell formed in the presence of FEN (the top center panel of FIG. 16) is less conformal than without API (lower center panel of FIG. 16), suggesting that FEN may end up in the shell after drying. Further, producing particles from a mixture of only ALG and PVA does not yield a shell (lower right panel of FIG. 16). The reduced porosity of this control (inset of lower right panel of FIG. 16) is believed to result from the significant excess of PVA in the continuous phase. This result suggests that only the PVA located at the nanoemulsion interface within the cross-linked polymer matrix leads to shell formation after the solvent and water are evaporated. Interestingly, while the dispersed phase is more viscous when saturated with API, which should lead to larger nanoemulsions, formulations with FEN have smaller diameter nanoemulsions (see Table 2) and subsequently smaller pore sizes (compare insets of the top right and lower center panels of FIG. 16). This suggests that FEN may act as a co-surfactant to stabilize a larger oil-water surface area, which is also consistent with the hypothesized integration of FEN into the PVA shell, when present.

TABLE 2
Comparison of the dynamic light scattering (DLS)
measurements between formulations with FEN and
those without FEN. Dispersed phase is emulsified in 2%
w/v ALG aqueous solution containing 1% w/v PVA.

| Volume | Dispersed | Dispersed |
|---|---|---|
| fraction of | phase with | phase |
| dispersed | FEN (C_{FEN} = | without |
| phase (φ) | C_{satFEN}) | FEN (control) |
| 20.0% | Diameter: 495 nm | Diameter: 1090 nm |
| | PDI: 0.253 | PDI: 0.317 |
| 30.0% | Diameter: 560 nm | Diameter: 1030 nm |
| | PDI: 0.255 | PDI: 0.313 |

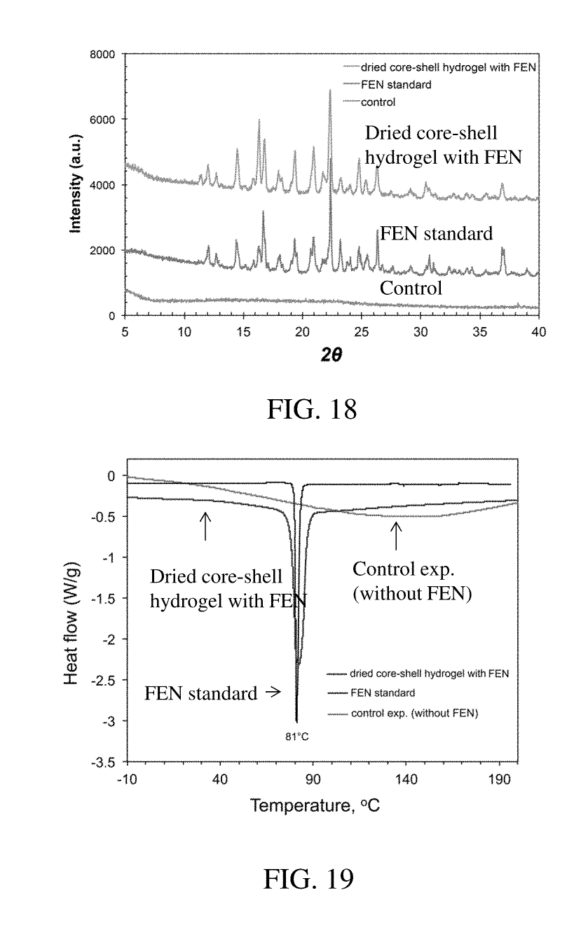

To investigate the crystallinity of the API nanocrystals embedded in the dried core-shell hydrogels, powder X-ray diffraction (PXRD) and differential scanning calorimetry (DSC) were performed (FIG. 18 and FIG. 19). In FIG. 18, XRD peaks produced by a typical dried core-shell hydrogel formulation (φ=30% dispersed phase with C_{FEN}=C_{satFEN} emulsified in 2% w/v ALG containing 1% w/v PVA emulsifier) match the peaks of the FEN standard. DSC measurements also exhibit a well-defined endotherm at approximately 81° C. corresponding to the melting point of fenofibrate. Both techniques thus demonstrate that encapsulated FEN nanoparticles are in the crystalline state within the final core-shell structure.

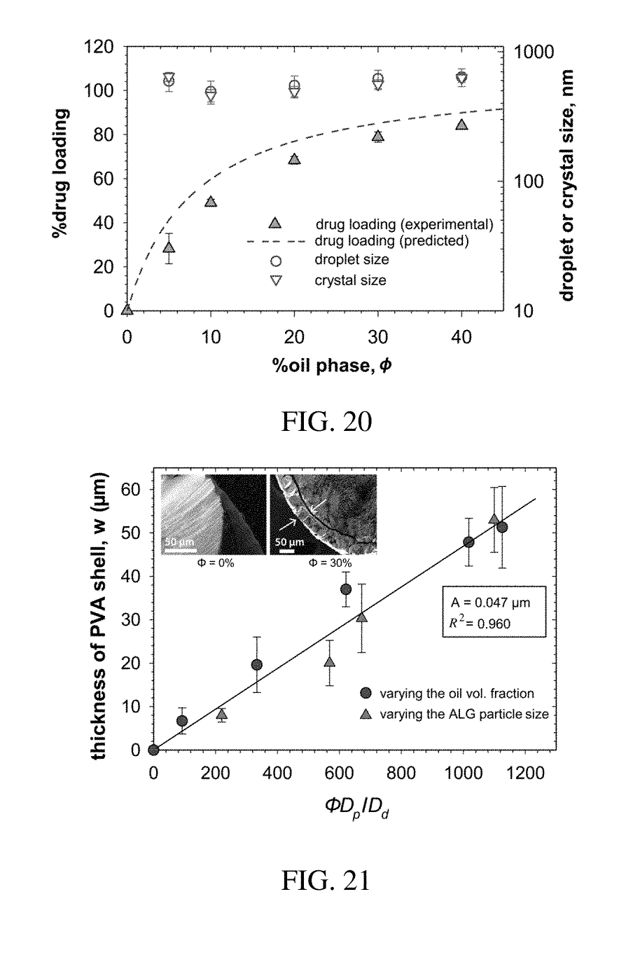

Controlling crystal is important in pharmaceutical manufacturing as it influences the pharmaceutical product performance characteristics such as dissolution rate and bioavailability. In FIG. 20, both mean nanoemulsion droplet size (<d_{d}>) measured by dynamic light scattering and mean crystal size (<d_{c}>) measured by SEM as a function of volume fraction (φ) of the dispersed phase are plotted. It should be noted that the volume fraction used here to prepare the emulsion laden hydrogels is limited to a maximum of 40%. Above this level, samples form a very foamy and viscous nanoemulsion solution that is difficult to handle. FIG. 20 shows that the average droplet size slightly increases from 460 nm to 630 nm with the increase of volume fraction from 10% to 40%, respectively. This variation of nanoemulsion droplet size corresponds with the shift in the emulsifier (PVA) to dispersed phase ratio. The obtained mean crystal size ranging from 500 nm to 640 nm (standard deviation of roughly 100 nm each) is nearly identical to the corresponding droplet size, indicating that the size of nanoemulsion droplets dictates the mean crystal size within the dried hydrogel matrix. This approach thus enables precise control over crystal size and morphology utilizing the elastic nature of the hydrogel matrix and nano-confinement. Such control is impossible to achieve in traditional evaporation induced crystallization processes. Further, this is mechanistically different than other approaches that use emulsions to influence crystallization and formulation.

Easy manipulation of drug loading is also a powerful feature in pharmaceutical manufacturing since it directly correlates with the dosage of a final formulation. FIG. 20 also shows the percentage drug loading at different volume fractions of dispersed phase at the FEN saturation concentration. The amount of FEN embedded in composite hydrogels (% loading on dry basis) can be controlled by tuning the volume fraction of the dispersed phase. To validate the experimental results, the predicted drug loading capacity was estimated based on the saturation concentration of fenofibrate and the formulation concentrations of PVA and alginate according to

$$\% drug loading = ( 100 \% ) \frac{{C}_{FEN} V φ}{( {C}_{ALG} + {C}_{PVA} ) V ( 1 - φ ) + {C}_{FEN} V φ} \qquad ( 1 )$$

where C_{FEN}Vφ is the mass of embedded FEN in a hydrogel bead with volume V, C_{ALG} and C_{PVA} are the concentrations of alginate and PVA, respectively, and V(1−φ) represents the volume of the continuous phase in a bead. The measured drug loadings are found to be slightly lower than the expected values, which may be due to the loss of some nanoemulsion droplets from hydrogel beads during the cross-linking and washing steps. However, adjustable and high loadings up to 83% can be achieved with relatively narrow distributions (<5%). This is the first demonstration of producing nanocrystals of a poorly soluble API with such a high loading using composite hydrogels.

To demonstrate the ability to design specific core-shell structures with this composite system, a scaling law for the outer PVA shell thickness as a function of the formulation parameters was developed. Experimentally, it was observed that the shell thickness can be tuned by (1) varying the overall hydrogel particle size at constant nanoemulsion volume fraction or (2) varying the dispersed phase volume fraction at constant PVA concentration in the continuous phase. Following method (1), core-shell hydrogel particles ranging from 430 μm to 2100 μm are prepared at constant nanoemulsion volume fraction (φ=30%) using different needle gauges while encapsulated nanocrystals of the API maintain the same size (˜620 nm). By method (2), the emulsifier/oil ratio is not maintained constant in all experiments, resulting in a variation of droplet sizes. Furthermore, formulations with a higher volume fraction of the dispersed phase yield hydrogel particles of increased size. Hence, using method (2) each preparation (data point in FIG. 21) will have a different nanoemulsion volume fraction, nanoemulsion droplet size, and alginate particle size.

Assuming only PVA from the liquid-liquid interfaces in the nanoemulsion migrate to the bead surface, the thickness of the shell is expected to scale as:

$$w = A ( \frac{φ {D}_{p}}{{D}_{d}} ) \qquad ( 2 )$$

where w is thickness of PVA layer, Dₚ is the diameter of the core-shell particle, D_{d} is the diameter of emulsion droplets and φ is the volume fraction of dispersed phase. The pre-factor, A, in Eq. (2) has dimensions of length and is related to the adsorption cross-section, molecular weight and amorphous density of PVA. FIG. 21 shows that the data for PVA shell thickness collapse onto a master curve and is linearly proportional to the dimensionless group

$$[ ( \frac{φ {D}_{p}}{{D}_{d}} ) ] .$$

The best fit to the experimental data produced a slope of 0.047 microns, which (combined with other known parameters) yields a value of 8.63 nm² for the adsorption cross-section of PVA. As shown in FIG. 21, the thickness of the PVA shell can be increased up to approximately 60 microns through either of the above experimental approaches by appropriately changing any of the three parameters involved. This scaling study shows the versatility of our core-shell composite hydrogels as drug delivery vehicles.

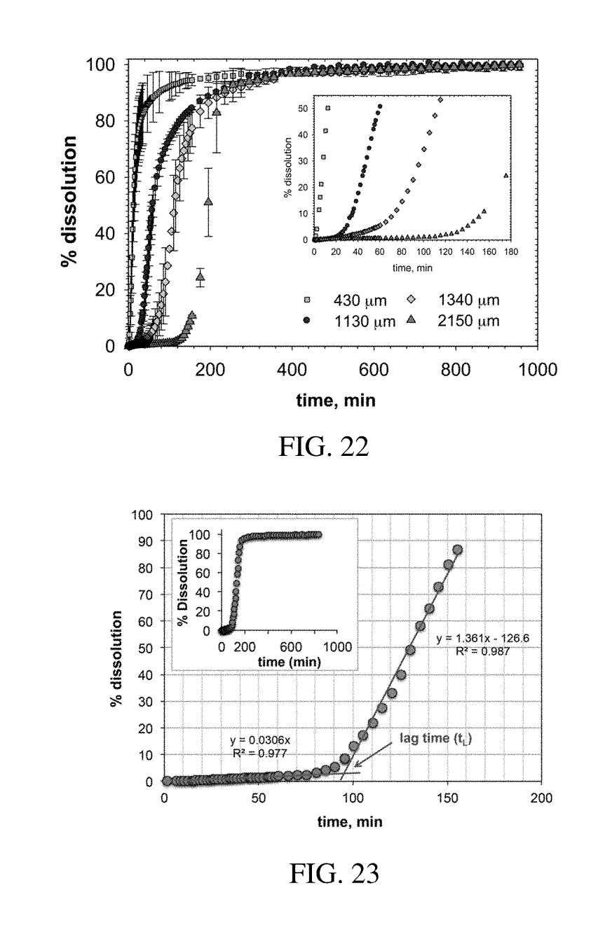

Simultaneously exploiting porous confinement in the core of the composite particles to control FEN crystal size and the tunable polymeric shell thickness, it is possible regulate the release/dissolution profile of embedded API. The in vitro dissolution profiles of FEN from the composite core-shell hydrogels prepared by method (1) at a volume fraction of φ=30% are plotted in FIG. 22. The figure demonstrates that drug is released slower from larger core-shell particles. The dissolution profiles of the larger particles also highlight the presence of two phases, a slow and fast region, of drug release. Initially, FEN release is extremely slow until a certain time denoted as the lag time, determined by the intersection of the linear extrapolation of the initial slow dissolution region (over which less than 5% of the drug dissolution occurs) and the subsequent fast dissolution region (see FIG. 23). The delay in dissolution is due to the presence of a PVA shell on the composite hydrogels that prevents the FEN nanocrystals contained in the core from being dissolved. As the ALG beads became larger, the lag time of drug release was observed to increase. This indicates that the lag time is greatly influenced by the thickness of the PVA shell on the composite hydrogels. However, in the second phase of drug dissolution, FEN was released very rapidly from all composite hydrogel formulations. Almost 80% of drug dissolution occurs within 30 to 100 min, depending on the ALG bead size. In particular, hydrogel beads that are smaller in size (˜430 μm) have no significant lag time and the release is very fast (80% drug dissolved within 30 min).

The characteristic features of the dissolution profiles presented in FIG. 22 demonstrate that API release from our composite particles is a two-part process. With reference to the final form of the core-shell particles shown in FIG. 15, the PVA shell is removed before the core is infiltrated with the suspending medium to initiate nanocrystal dissolution. Following solubilization of the API, it diffuses a distance several orders of magnitude larger than the crystal itself to reach the surface of the porous hydrogel matrix before it is finally released. The timescale of this complete process can be understood by modelling the underlying mechanisms of mass transport.

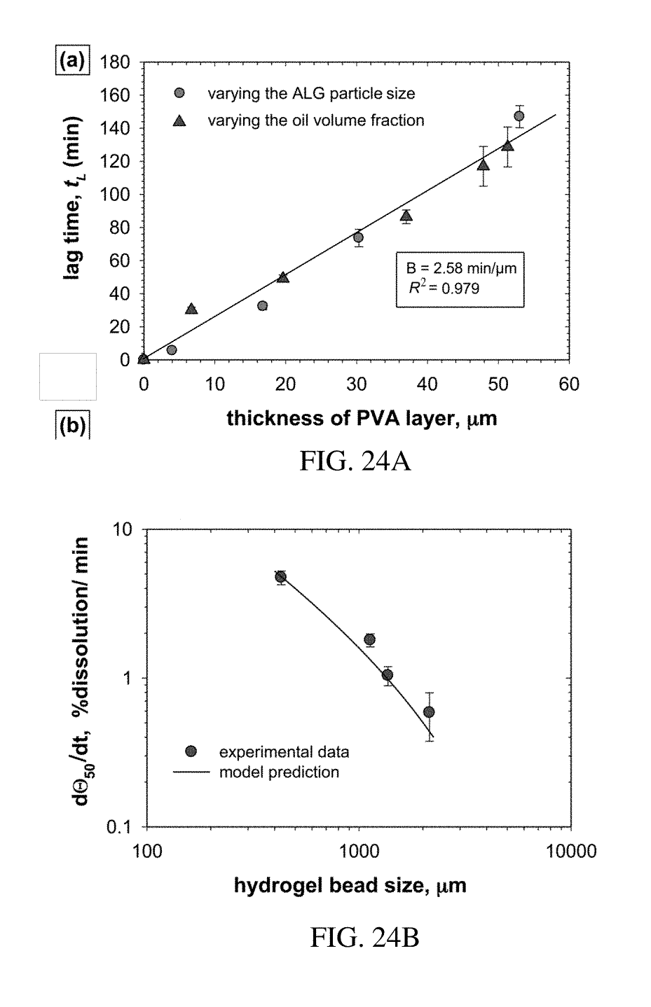

To quantify the correlation between the lag time (from FIG. 22) and the thickness of the PVA layer (measured from SEM), these values are plotted relative to each other in FIG. 24A. The comparison of experimental results shows that lag time and shell thickness are linearly correlated with a proportionality constant of about 2.58 min/μm. The length of this lag time, t_L, can be predicted quantitatively by modeling the dissolution kinetics of the PVA layer of thickness, w. By developing a quantitative model, the necessary particle properties can be determined a priori for the synthesis of a vesicle with desirable dissolution characteristics.

The dissolution time of the PVA shell can be calculated once an analytical form of the diffusive PVA concentration profile in the surrounding fluid is derived. Assuming a pure, uniform solid shell, removal of PVA can be driven by convective mass transport in the stirred vessel. The rate limiting step of such a system is dissolution of the solid as indicated by a large Biot number, Biₘ, which is the ratio of the rates of convection and diffusion. Mass transport in this system is represented by a purely diffusive boundary layer at the shell surface over which a pseudo steady-state diffusion profile is formed. The concentration at the particle surface is set by the solubility of PVA, Cₛ, and the concentration at the boundary layer interface is dictated by the conservation of mass flux from diffusion inside the boundary layer and convection outside. Additionally, the concentration dependence of PVA diffusivity can be accounted for to accurately represent the diffusive flux. A power law dependence of the form D(C)=αD₀C^{-γ} was adopted, where D₀ and C are the bare diffusion and concentration of PVA (in units of g/L), respectively. The experimental studies with PVA in the semi-dilute regime suggest a value of α=0.005 and the molecular weight dependence of the power law exponent for polyelectrolytes yields γ=0.0061M_{w}^{0.35}=0.298. Implementing the concentration dependence of diffusivity and the mixed boundary conditions described above while solving Fick's second law results in an analytical solution for the steady-state concentration profile.

By balancing the mass flux into the boundary layer (using the PVA concentration profile) with the loss of mass from the shell surface, the total time to remove the shell can be estimated according to

$${t}_{L} = \frac{{ρ}_{s}}{h {C}_{δ}} w = Bw \qquad ( 3 )$$

where C_{δ} is the concentration of PVA at the boundary layer interface. The slope, B, is a function of C_{δ} as well as the density of amorphous PVA, ρₛ, and the convective mass transport coefficient, h, resulting from the vessel stirring conditions. The value of C_{δ} is determined as the eigenvalue of (1−γ)(1+1/Biₘ)(C_{δ}/Cₛ)=1−(C_{δ}/Cₛ)^{1-γ}, which is the analytical solution of the PVA concentration profile at the boundary layer interface. For the range of particle sizes studied here, a value of C_{δ}=0.52c, was determined for all corresponding values of Biₘ, (with variations less than 0.1%). Using characteristic PVA parameters of D₀=3×10⁻¹¹ m²/s, ρₛ=1.27 g/mL, Cₛ=0.8 g/mL, the proportionality constant is calculated as B=2.5 min/μm, which agrees well with the fitted value in FIG. 24A. Hence, shell dissolution is a diffusion limited process and Eq. (3) provides an accurate estimate of the necessary shell thickness for a desired lag time in API dissolution.

The dependence of the dissolution rate (region 2 of API release in FIG. 22) on drug loading and particle size can also be estimated by a simple mass transport model. Under United States Pharmacopeia (USP) dissolution experimental conditions, diffusion within the bead is the rate limiting step. Thus, the flux of drug into solution is proportional to the drug concentration at the bead surface and the dissolution rate can be estimated by modeling the concentration profile inside the bead. The maximum rate (in the experimentally measured units of percent of total dissolved solid per minute) can be estimated according to

$$\frac{\partial }{\partial t} ( \frac{C}{{C}_{total}} ) = ( 100 \% ) \frac{6 {hC}_{max} ( \tilde{r} = 1 , τ )}{φ {D}_{p} {C}_{sat}} \qquad ( 4 )$$

where Cₜₒₜₐₗ is the final concentration of API in the bulk and Cₘₐₓ is the largest estimated FEN concentration at the bead surface. Estimates of Cₘₐₓ are calculated by treating FEN dissolution as a two-step process involving nano-crystal dissolution then diffusion to the bead surface. The time scales of these processes differ by orders of magnitude and are therefore decoupled. Given that the nanocrystals are well dispersed, the hydrogel core is treated as a homogeneous sphere with a transient radial concentration profile. The “initial” concentration of the sphere evolves over time according to the transient radial concentration profile at half the average distance between the nanocrystals, which are treated as spheres with a constant surface concentration set at C_{satFEN}. Analytical solutions exist for both of these forms of transient diffusion. The maximum surface concentration is the product of these two mass transport processes, which results in a power law dependence on Biₘ, (i.e., bead diameter) according to Cₘₐₓ∝Biₘ^{C(φ)}, where the pre-factor and exponent are functions of volume fraction. Dissolution rates estimated using these values of Cₘₐₓ are shown in FIG. 24B compared to experimental results (at φ=30%), which show excellent agreement. The accuracy of our dissolution model indicates that the bead size controls the rate (and thus time) of dissolution, which results from the controlled crystallization on the nano-scale. This model is sufficiently general to represent the release of nanocrystals of any API. By removing crystal dissolution as a mass transport barrier, our composite hydrogels offer a method of rapid drug release coupled with a well-controlled lag time, if desired.

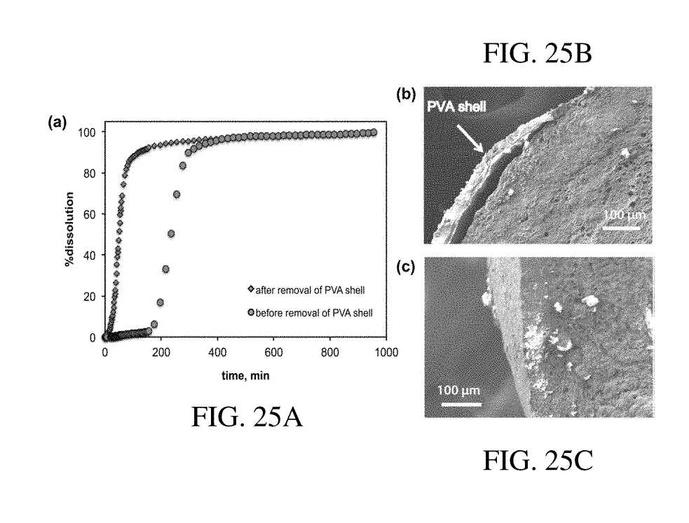

To further investigate the influence of the shell on the release behavior of FEN nanocrystals embedded inside the core, dissolution measurements were carried out using composite hydrogels without a shell. The PVA shell was removed by washing the composite hydrogels with water and then drying again at 60° C. The ALG beads without the PVA layer exhibit faster release profiles without any lag time (FIG. 25). This result confirms that the densely packed PVA shell may provide a diffusion barrier for the guest molecules embedded inside the confined environment of ALG core. Further, it offers additional customization of the final release profile by completing a simple post-production washing step of the initial core-shell beads.

This study demonstrates that the drug dissolution patterns from core-shell composite hydrogels can be modulated through incorporating a lag phase of pre-established duration in their release profile by altering the thickness of a PVA shell. Properly modulated lag phases prior to drug release may be advantageous in a number of instances. One example is avoiding undesired drug-drug interactions in the gastrointestinal track, which will comply with chronotherapeutic needs and thus improve the overall patient experience. Overall, this formulation methodology provides a facile way to tailor drug release kinetics by taking advantage of the tunable properties of core-shell microstructured hydrogels. Furthermore, because of their bio-friendly nature, and the adjustable nanocrystal size and drug loading capacity, the proposed composite core-shell hydrogels could potentially be integrated into a final solid formulation form (e.g. capsules or tablets) for oral delivery.

### CONCLUSION

In summary, composite hydrogel microparticles with core-shell structures and their use for formulating nanocrystals of water insoluble APIs are reported. Higher drug loading embedded inside a hydrogel core matrix with improved dissolution kinetics utilizing the advantages of a core-shell microstructure is demonstrated. The incorporation of hydrophobic nano-domains carrying the APIs inside the hydrogel matrix represents a promising formulation approach for improving the solubility and bioavailabilty of water-insoluble APIs. Furthermore, it is possible to independently control the shell and crystal sizes to engineer a core-shell microstructure to quantitatively tune the drug dissolution kinetics, providing a versatile drug delivery vehicles. The novel approach of simultaneous controlled crystallization and formulation could potentially circumvent several energy intensive top-down processes in traditional manufacturing. With the ability to produce and formulate nanometer sized crystals with controlled size, adjustable drug loading capacity and tunable release properties, the proposed core-shell hydrogels could potentially serve as a final drug formulation that is even amenable to continuous manufacturing. More generally, the versatility and orthogonal accessibility of the composite core-shell hydrogels opens up a wide range of applications such as the design of multidrug therapies, the preparation of designer foods, delayed/pulsatile drug delivery and cell encapsulation. The proposed approach is not limited to only pharmaceutical products, but can also be useful to production of a wide range of crystalline nanomaterials.

### Experimental Section

### Materials:

Fenofibrate (CAS no. 49562-28-9, >99% pure), anisole (CAS no. 100-66-3, >99% pure), calcium chloride (CAS no. 10043-52-4, >93% pure), sodium dodecyl sulfate (CAS no. 151-21-3, >99% pure), and polyvinyl alcohol (Mowiol® 8-88, M_{w} ˜67,000, CAS no. 9002-89-5) were purchased from Sigma-Aldrich and used as received. Sodium alginate (CAS no. 9005-38-3), a polysaccharide consisting of approximately 61% mannuronic (M) and 39% guluronic (G) acid was also purchased from Sigma. 14, 15, 20, and 22 gauge needles were purchased from Nordson EFD. DI water was used throughout the experiments.

### Nanoemulsion Preparation and Characterization:

To prepare the nanoemulsions, a pre-emulsion was first generated by adding the dispersed phase (anisole containing a saturated concentration of FEN) into the continuous phase (2% w/v sodium alginate solution containing 1% w/v PVA) using a magnetic stirrer bar for 30 minutes at 700 rpm. The dispersed phase of anisole saturated with FEN was prepared by bringing excessive amounts of FEN in contact with anisole to establish solid-liquid equilibrium at room temperature. The mother batch was allowed to equilibrate for 24 h at room temperature at 200 rpm stirring speed.

The pre-emulsion was then ultrasonicated in 5 mL aliquots at 30% amplitude in an ultrasonicator with a 24 mm diameter horn (from Cole Parmer) at a frequency of 20 kHz. Nanoemulsion droplet sizes were measured via dynamic light scattering (DLS) using a Wyatt Technology DynaPro NanoStar Instrument. Samples were diluted to φ=0.001 in deionized water. Autocorrelation functions were measured at a scattering angle of 90° and a temperature of 25° C. Three independent measurements were taken for each sample. Each DLS measurement was taken over a 50 second acquisition. Size and polydispersity were extracted from raw DLS data using second order cumulant analysis.

### Procedure for Core-Shell Hydrogels Preparation:

To prepare the core-shell hydrogel particles, uncross-linked nanoemulsion solution contained in a 5-mL syringe was dripped into a CaCl₂ solution using a microfluidic positive displacement pump (KD Scientific 110). For the core-shell particles obtained at various volume fractions of dispersed phase (φ), a 15 gauge stainless steel blunt-tip needle was used for dripping, unless specified otherwise. The dripping height was set to 10 cm and the dripping bath containing 6% w/v CaCl₂ was stirred with a magnetic stirrer at 100 rpm. The resulting hydrated hydrogel particles were then washed by exchanging the calcium chloride cross-linking solution five times with deionized water. They were then filtered using a Buchner funnel and rinsed once more with deionized water. The nanoemulsion laden cross-linked hydrogels were then allowed to be dried in an oven at 60° C. for 2-4 days, leading to the formation of hydrogel materials with core-shell microstructures. The sizes of resulting composite core-shell hydrogels ranged from 1200 microns to 2000 microns depending on the volume fraction of dispersed phase used in the experiments. At constant dispersed phase (φ=30%), various sized core-shell hydrogel particles were also generated following the above experimental procedures, but using different needle sizes. For example, 1130 micron, 1340 micron, and 2150 micron sized hydrogel beads were prepared using 22 G, 20 G and 14 G needles, respectively. However, to produce the 430 micron sized particles, centrifugal synthesis approach was used.

430 μm sized core-shell composite hydrogels were prepared using a centrifugal synthesis method implementing a nozzle-in-centrifuge apparatus. To prepare the nozzle-in-centrifuge apparatus, falcon tubes (50 mL) were utilized as supports (outer chamber) for the syringe and needle (inner chamber) while also serving as the CaCl₂ bath. The volume of the CaCl₂ solution was used to adjust the distance between the needle tip and the CaCl₂ bath (H). Syringes (10 mL) were cut to shape by hand and secured through a punctured tube cap using hot melt adhesive to maintain needle positioning. 20 gauge stainless steel blunt-tip needles were used in this experiment. This nozzle-in-centrifuge apparatus provides accessibility to interchange needle tips and other component specifications.

To prepare small core-shell composite hydrogel beads, 2 mL of uncross-linked alginate containing nanoemulsion solution (φ=30% dispersed phase with C_{FEN}=C_{satFEN} emulsified in 2% w/v ALG containing 1% w/v PVA) and 7.5 mL of CaCl₂ (6.0% w/v) solutions were injected into the inner and outer chambers, respectively, prior to centrifugation. The cap securing the nanoemulsion solution (containing the syringe and nozzle) is fixed at a height such that the distance H between the nozzle tip and the CaCl₂ bath was 10 mm. The length of the needle used as a nozzle was 18 mm. Centrifugation was imposed at 1000 rpm for 60 seconds. The nanoemulsion laden cross-linked hydrogels were then collected, washed several times with water, and dried in an oven at 60° C. for 2-4 days to obtain the core-shell composite particles.

### Loading Measurements:

The nanoemulsion laden hydrogels are synthesized with different nanoemulsion volume fractions ranging between 10 and 40%. For each measurement of a given volume fraction, two batches of nanoemulsion laden hydrogels are prepared with the same method of emulsification followed by cross-linking. One is a reference batch without API and the other is a test batch carrying dissolved API at the saturation concentration. The samples from both batches (approximately 200 mg of ALG beads) are placed in a vacuum oven, dried over 3 days at 60° C. and then weighted. Drug loading is defined as the difference in weight between the dried test batch carrying API and the dried reference batch formulated without the API divided by the weight of dried test batch carrying API. Loading measurements were done in five replicates, the average of which are reported with sample standard deviations. The experimentally measured drug loading capacity is also compared to the predicted value calculated according to Eq. (1).

### Analysis of Core-Shell Hydrogel Materials:

The Dried Composite Core-Shell Hydrogels were analyzed by powder X-ray diffraction (PXRD) in reflectance mode (Panalytical X'pert MPD Pro). The samples were ground then placed on a zero background disk. The PXRD was operated at 40 kV, 30 mA, and at a scanning rate of 2°/min over the range of 2θ=10-40°, using Cu Kα radiation wavelength of 1.54 Å. Samples were also analyzed by differential scanning calorimetry (DSC) using TA Instruments (Q2000 DSC). 10-15 mg of sample was crimped in a sealed T-zero aluminum pan and heated at 10° C./min in the range of −20° C. to 250° C. using an empty sealed pan as a reference. Dry nitrogen was used as purge gas and the N₂ flow rate was 50 mL/min. Raman spectra of the composite hydrogel with FEN nanocrystals embedded within it were obtained with the aid of Raman spectroscopy coupled to a Leica optical light microscope. The Raman microscope (Kaiser Optical Systems, Inc.) was equipped with a 450-mW external cavity stabilized diode laser as the excitation source, operating at 785 nm. The size and morphology of the composite hydrogel particles and the embedded FEN crystals were characterized with high resolution scanning electron microscope (Zeiss HRSEM) at 5 kV accelerating voltage and at various magnifications. All samples were prepared on conventional SEM stubs with carbon tape and were coated with about 10-15 nm of Au—Pd by sputter coating. The images were later analyzed with ImageJ manually to calculate the mean particle and crystal size.

### Dissolution Experiments:

The in vitro dissolution of FEN from the prepared core-shell hydrogels was carried out using the standard USPII (paddle) apparatus at 37° C. and 75 rpm. The dissolution medium was 600 mL of 0.2 M phosphate buffer at pH 6.8, containing 0.72% w/v SDS. The pH value was chosen so that the hydrogels would be encouraged to swell and, with the uptake of water, consequently release their drug contents. Samples of dried composite hydrogel formulation (equivalent to about 12.5 mg of drug) was added to the dissolution media manually. Given the loading of FEN and the saturation solubility of FEN in the media, the mass of FEN added for a dissolution experiment was at least 3 times less than the mass of FEN required to saturate the media, thus maintaining sink conditions during dissolution experiments. The UV measurements were obtained using an automatic Varian UV-vis Cary 50 apparatus and in situ probe set. All reported measurements were repeated at least 3 times under identical conditions and averaged values are reported here.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method comprising:
forming an emulsion comprising a non-aqueous carrier containing a hydrophobic drug dispersed in an aqueous carrier, wherein the aqueous carrier comprises a first polymer, and wherein the emulsion further comprises poly(vinyl alcohol);
at least partially cross-linking the first polymer by exposing it to a cross-linking agent;
removing sufficient aqueous carrier and sufficient non-aqueous carrier, thereby crystallizing the drug as a dispersion of solid hydrophobic drug in a cross-linked matrix comprising the first polymer.

2. A method comprising:
forming an emulsion comprising a non-aqueous carrier containing a hydrophobic drug dispersed in an aqueous carrier, wherein the aqueous carrier comprises a first polymer, and wherein the emulsion further comprises a second polymer;
at least partially cross-linking the first polymer by exposing it to a cross-linking agent;
removing sufficient aqueous carrier and the non-aqueous carrier, thereby:
crystallizing the drug as a dispersion of solid hydrophobic drug in a cross-linked matrix comprising the first polymer, and
forming a composite core-shell particle, wherein the shell comprises the second polymer.

3. A composite core-shell particle comprising:
a core, which comprises a crystalline hydrophobic drug embedded in a cross-linked polymer; and
a shell, which comprises poly(vinyl alcohol).

4. A method as in claim 1, wherein the hydrophobic drug is dissolved in the non-aqueous carrier.

5. A method as in claim 1, wherein the first polymer comprises alginate.

6. A method as in claim 2, wherein the second polymer comprises poly(vinyl alcohol).

7. A method as in claim 1, wherein the hydrophobic drug comprises fenofibrate.

8. A method as in claim 1, wherein the crystallization step comprises forming crystals with a diameter of less than or equal to 0.65 microns.

9. A method as in claim 1, wherein after the removal step, particles are formed which comprise a weight percentage of the drug of at least 30%.

10. A method as in claim 1, wherein after the removal step, particles are formed which comprise a polymeric shell surrounding a cross-linked polymeric matrix.

11. A method as in claim 10, wherein, after the removal step, the cross-linked polymeric matrix comprises the crystalline drug.

12. A method as in claim 1, wherein the cross-linking step comprises the formation of ionic cross-links.

13. A method as in claim 12, wherein the ionic crosslinks comprise an alkaline earth metal cation.

14. A method as in claim 1, wherein the removal of the aqueous carrier and non-aqueous carrier comprises heating.

15. A method as in claim 1, wherein the removal of the aqueous carrier and the non-aqueous carrier occurs at a temperature of at least 60° C.

16. A method as in claim 1, wherein the emulsion comprises a dispersed phase volume fraction of less than or equal to 40%.

17. A method as in claim 1, wherein the emulsion is a nanoemulsion.

18. A method as in claim 17, wherein the nanoemulsion comprises droplets with a diameter of 0.65 microns.

19. A method as in claim 1, wherein the dispersion is a nanodispersion.

20. A method as in claim 2, wherein the core further comprises water.
